(19) United States
(12) Patent Application Publication
(10) Pub. No.: US 2026/0199427 A1
(43) Pub. Date: Jul. 16, 2026

(54) METHODS FOR THE PREVENTION OR TREATMENT OF ANTHRAX INFECTION

(71) Applicant: Cumberland Pharmaceuticals Inc., Nashville, TN (US)

(72) Inventors: Leo PAVLIV, Cary, NC (US); Ines MACIAS-PEREZ, Mt. Juliet, TN (US)

(73) Assignee: Cumberland Pharmaceuticals Inc., Nashville, TN (US)

(21) Appl. No.: 19/560,105

(22) Filed: Mar. 8, 2026

Related U.S. Application Data
(63) Continuation of application No. 17/992,470, filed on Nov. 22, 2022, now abandoned.
(60) Provisional application No. 63/283,068, filed on Nov. 24, 2021.

(51) Int. Cl. A61K 38/14; A61P 31/04

(52) U.S. Cl. CPC A61K 38/14; A61P 31/04

(57) ABSTRACT

Methods for preventing or treating a Bacillus anthracis (anthrax) infection in a subject, such as a mammal, by administering to the subject telavancin or a pharmaceutically-acceptable salt thereof.

### CROSS-REFERENCE TO RELATED APPLICATIONS

This application claims priority to and the benefit of U.S. provisional application No. 63/283,068, filed Nov. 24, 2021, which is hereby incorporated by reference in its entirety.

### FEDERALLY SPONSORED RESEARCH OR DEVELOPMENT

This invention was made with Government support under Contract Nos. HHSN2722017000401 and 75N93019D00005, awarded by the Division of Microbiology and Infectious Diseases, National Institute of Allergy and Infectious Diseases, National Institutes of Health, and U.S. Department of Health and Human Services. The Government has certain rights in the invention.

### BACKGROUND

The causative agent of anthrax is Bacillus anthracis, a Gram-positive spore-forming rod-shaped bacterium. This bacterium can infect humans by cutaneous, gastrointestinal, inhalation and injection routes. B. anthracis exists in two forms, vegetative cells (inside the host) and spores for persistence in the soil or environment. In the soil, B. anthracis is generally found in endospore form where it can remain viable for decades. As B. anthracis forms spores that can be aerosolized and sprayed to spread disease, the potential use of this bacterium as a bioterrorism agent has long been suspected. However, the events in 2001 have confirmed that bioterrorism is no longer a threat but a reality. Owing to its highly pathogenic nature and spore forming capability, B. anthracis is considered as one of the most important biological warfare agents. The Center for Disease Control and Prevention recognizes this bacterium as a Category A agent with recognized bioterrorism potential.

The possibility of emerging natural resistance or “engineered” resistance in B. anthracis is also an area of great concern (see, e.g., Heine et al., Antimicrob. Agents Chemother., 51(4): 1373-1379 (2007); Heine et al., Antimicrob. Agents Chemother., 61(9):e00788-17 (2017)). For example, although penicillin has long been considered the treatment of choice for anthrax, numerous reports $of β-lactamase-producing strains, and treatment failures have appeared in the literature. Additionally, two open reading frames coding for β-lactamases have been identified in the B. anthracis genome. Several reports of B. anthracis resistance to ciprofloxacin, macrolides, and tetracyclines have appeared in the literature. With the added concern of engineered resistance in a biological threat setting, there is an urgent need for new antibiotics to counter the emergence of new bacterial pathogens and resistance to existing antibacterial drugs.

### SUMMARY OF THE INVENTION

Disclosed herein are methods for treating a Bacillus anthracis (anthrax) infection in a subject, comprising administering to the subject a pharmaceutical composition consisting of a therapeutically effective amount of telavancin or a pharmaceutically acceptable salt thereof and at least one pharmaceutical excipient.

Also disclosed are methods for preventing a Bacillus anthracis (anthrax) infection in a subject at risk of exposure to Bacillus anthracis, comprising administering to the subject a pharmaceutical composition consisting of a therapeutically effective amount of telavancin or a pharmaceutically acceptable salt thereof and at least one pharmaceutical excipient.

Also disclosed are methods for treating or preventing a Bacillus anthracis (anthrax) infection in a mammal, comprising intravenously administering to the mammal a pharmaceutical composition comprising a therapeutically effective amount of telavancin hydrocholoride and at least one pharmaceutical excipient selected from the group consisting of: 2-hydroxypropyl-β-cyclodextrin, mannitol, sodium hydroxide, hydrochloric acid, 5% Dextrose Injection, Sterile Water for Injection, 0.9% Sodium Chloride Injection, and combinations thereof.

Additional aspects and advantages of the embodiments disclosed herein will be set forth in part in the description that follows and, in part, will be discemable from the description, or may be learned by practicing or performing the disclosed embodiments. The aspects and advantages will be realized and attained by means of the elements and combinations particularly pointed out in the appended claims.

It is to be understood that both the foregoing general description and the following detailed description are exemplary and explanatory only and are not restrictive of the scope of the claim appended hereto.

### BRIEF DESCRIPTION OF THE DRAWINGS

FIG. 1 shows the preparation of a 96-well round bottom plate with various dilutions of treatment and control solutions.

FIG. 2 shows the survival results for the treatment groups over the 14-day post-challenge period.

FIG. 3 shows the average weight (with standard error bars) for each treatment group during the post-challenge period.

FIG. 4 shows the maximum percent weight loss for the antibiotic-treated groups and saline treated group.

FIG. 5A shows the average level of bacteremia for the antibiotic-treated groups and saline treated group.

FIG. 5B shows the average level of bacteremia for the two antibiotic-treated groups.

FIG. 6A shows the average bacterial load in the lung tissue for the antibiotic-treated groups and saline treated group.

FIG. 6B shows the average bacterial load in the lung tissue for the two antibiotic-treated groups.

FIG. 7 shows the serum anti-PA IgG response in the antibiotic-treated groups and saline treated group.

FIG. 8a shows the survival results for the treatment groups over the 14-day post-challenge period.

FIG. 8b shows the survival results for the treatment groups over the 14-day post-challenge period where animals 6083, 6063, and 6066 are removed.

FIG. 9a shows the average daily weights for each treatment group during the post-challenge period.

FIG. 9b shows the average daily weights for each treatment group during the post-challenge period where animals 6083, 6063, and 6066 are removed.

FIG. 10a shows the average anti-PA antibody responses of the antibiotic-treated groups post-challenge.

FIG. 10b shows the average anti-PA antibody responses of the antibiotic-treated groups post-challenge where animals 6066 and 6063 are removed.

### DETAILED DESCRIPTION OF THE INVENTION

The disclosed embodiments relate to methods for preventing or treating a Bacillus anthracis (anthrax) infection in a subject in need thereof by administering to the subject telavancin or a pharmaceutically-acceptable salt thereof.

It is to be understood that the invention is not limited to the particular embodiments of the invention described herein, as variations of the particular embodiments may be made and still fall within the scope of the appended claims. It is also to be understood that the terminology employed herein is for the purpose of describing particular embodiments and is not intended to be limiting. Instead, the scope of the present invention will be established by the appended claims.

Further, recitation of ranges of values are merely intended to serve as a shorthand method of referring individually to each separate value falling within the range, unless otherwise indicated herein, and each separate value is incorporated into the specification as if it were individually recited herein. Where a range of values is provided, it is to be understood that each intervening value, to the tenth of the unit of the lower limit, unless the context clearly dictates otherwise, between the upper and lower limit of that range and any other stated or intervening value in that stated range is encompassed within the invention. The endpoints of all ranges are included within the range and independently combinable. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included.

All methods described herein can be performed in a suitable order unless otherwise indicated herein or otherwise clearly contradicted by context. The use of any and all examples, or exemplary language (e.g., “such as”), is intended merely for illustration and does not pose a limitation on the scope of the invention unless otherwise claimed. No language in the specification should be construed as indicating any non-claimed element as essential to the practice of the invention.

Unless defined otherwise, all technical and scientific terms used herein have the same meaning as commonly understood by one of ordinary skill in the art to which the disclosed embodiments belong. Any methods and materials similar or equivalent to those described herein can also be used in the practice or testing of the disclosed embodiments described herein. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

### Definitions

When describing this invention, the following terms have the following meanings unless otherwise indicated.

The term “bi-weekly” refers to a frequency of every 13-15 days, the term “monthly” refers a frequency of every 28-31 days and “bimonthly” refers a frequency of every 56-62 days.

As used herein the term “consisting essentially of” refers to a composition whose only active ingredient is telavancin or a pharmaceutically-acceptable salt thereof, however, other components may be included in the composition for stabilizing, preserving, administering, facilitating the release of the active ingredient, etc., but are not involved directly in the therapeutic effect of the telavancin or a pharmaceutically-acceptable salt thereof.

As used herein, the term “contacting” is meant to broadly refer to bringing a bacterial cell and a molecule of telavancin into sufficient proximity that the telavancin can exert an effect on the bacterial cell. The telavancin may be transported to the location of the bacterial cell, or the telavancin may be situated in a location to which the bacterial cell travels or is brought into contact. The skilled artisan will understand that the term “contacting” includes physical interaction between telavancin and a bacterial cell, as well as interactions that do not require physical interaction.

The term “free base equivalent(s)” means the amount of free base in an acid addition salt of the free base and an acid (i.e., the amount of free base if the acid addition salt form was converted to the free base form). For example, one gram of telavancin dihydrochloride salt (MW=1828.5) contains 0.96 grams of telavancin (MW=1755.63) and 0.04 grams of hydrogen chloride (MW=36.46); or 0.96 grams of telavancin free base equivalents.

As used herein, the terms “inhibit”, “inhibiting” and “inhibition” have their ordinary and customary meanings, and include one or more of inhibiting colonization of B. anthracis, inhibiting growth of a vegetative form of B. anthracis, inhibiting a function of a vegetative form of B. anthracis, inhibiting propagation of a vegetative form of B. anthracis, inhibiting B. anthracis sporulation, inhibiting activation of a B. anthracis spore, inhibiting germination of a B. anthracis spore, and inhibiting outgrowth of a B. anthracis spore. Such inhibition is an inhibition of about 1% to about 100% of the particular activity versus activity in the absence of the telavancin. For instance, the inhibition is an inhibition of 100%, 99%, 98%, 97%, 96%, 95%, 90%, 80%, 70%, 60%, 50%, 40%, 30%, 20%, 10%, 5% or 1% of the activity versus activity in the absence of telavancin. As used herein, the inhibition lasts at least 0.5, 1, 2, 3, 4, 5, 6, 7, 10, 12, 15, 20, 25, 30, 35, 40, 45, 50, 55, 60, or more days after administration of telavancin or a pharmaceutically-acceptable salt thereof.

The term “pharmaceutically-acceptable salt” means a salt that is acceptable for administration to a human subject (e.g., salts having acceptable mammalian safety for a given dosage regime). Representative pharmaceutically-acceptable salts of telavancin include acid addition salts of telavancin with acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

As used herein, a “subject” refers to an animal, such as a mammal or a human. The subject may have an asymptomatic B. anthracis infection, a symptomatic B. anthracis infection, may be at risk for developing a B. anthracis infection, or may be at greater risk than the general population for developing a B. anthracis infection. Examples of subjects having a higher risk for B. anthracis infection include subjects with impaired immune function (e.g., immunoglobulin deficiency, splenic dysfunction, splenectomy, HIV infection, impaired leukocyte function, hemoglobinopathies), the elderly, people with certain malignancies (e.g., multiple myeloma, chronic lymphocytic leukemia, lymphoma), people at increased occupational risk (e.g., public services workers, such a fire, water, sanitary, police, medical, and laboratory workers, hospital workers, public servants such as mail-room workers and government employees, members of the press and media), people in closed populations (e.g., prisons, military, nursing homes) and those that have immunological deficiencies that might enhance their susceptibility to bacterial infection.

As used herein, “spore” refers to both the conventionally used terms “spore” and “endospore.”

The term “telavancin” means the compound N³″-[2-(decylamino)ethyl]-29-[[(phosphonomethyl)amino]methyl]vancomycin having the formula:

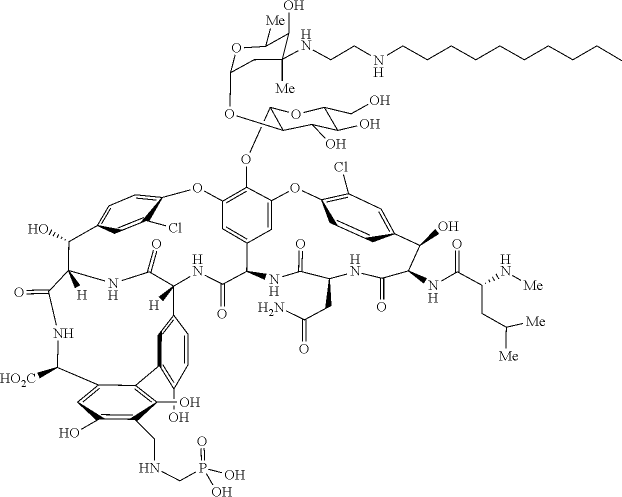

The term “telavancin hydrochloride” means any hydrochloride salt of telavancin, including, for example, the mono-, di- and trihydrochloride salts of telavancin and mixtures thereof (e.g., x·HCl, where x is 1 to 3).

As used herein, the terms “treating” and “treatment” have their ordinary and customary meanings to reduce the incidence or progression of disease following exposure to B. anthracis, such as, for instance, by ameliorating a symptom of B. anthracis infection in a subject, blocking or ameliorating a recurrence of a symptom of B. anthracis infection in a subject, decreasing in severity and/or frequency a symptom of B. anthracis infection in a subject, stasis, decreasing, or inhibiting growth of a vegetative form of B. anthracis in a subject, inhibiting B. anthracis sporulation, inhibiting activation of a B. anthracis spore in a subject, inhibiting germination of a B. anthracis spore in a subject, and inhibiting outgrowth of a B. anthracis spore in a subject. Treatment means ameliorating, blocking, reducing, decreasing or inhibiting by about 1% to about 100% versus a subject to which telavancin or a pharmaceutically-acceptable salt thereof has not been administered. The ameliorating, blocking, reducing, decreasing or inhibiting is 100%, 99%, 98%, 97%, 96%, 95%, 90%, 80%, 70%, 60%, 50%, 40%, 30%, 20%, 10%, 5% or 1% versus a subject to which telavancin or a pharmaceutically-acceptable salt thereof has not been administered. The treatment may begin prior to, concurrent with, or after the onset of a clinical symptom of an infection.

As used herein, the terms “preventing” and “prevention” have their ordinary and customary meanings, to reduce the incidence or progression of disease in a subject at risk of exposure to B. anthracis, such as, for example, by preventing colonization of B. anthracis in a subject, preventing an increase in the growth of a population of B. anthracis in a subject, preventing activation, germination or outgrowth of B. anthracis spores in a subject, preventing sporulation of B. anthracis in a subject, preventing development of a disease caused by B. anthracis in a subject, and/or preventing symptoms of a disease caused by B. anthracis in a subject. As used herein, the prevention lasts at least 0.5, 1, 2, 3, 4, 5, 6, 7, 10, 12, 15, 20, 25, 30, 35, 40, 45, 50, 55, 60, or more days after administration of telavancin or a pharmaceutically-acceptable salt thereof.

The term “therapeutically effective amount” means an amount sufficient to effect treatment and/or prevention when administered to a subject in need thereof. The therapeutically effective amount will vary depending on, for instance, the characteristics of the subject, the disease state being treated or prevented, the severity of the affliction, the extent of the risk of exposure, the period of time since infection or until risk of exposure, the formulation, and/or the manner of administration.

### Telavancin Drug Substance

Telavancin or any pharmaceutically-acceptable salt of telavancin can be employed in the methods described herein. In one embodiment, telavancin hydrochloride is used. Telavancin hydrochloride is an off-white to slightly colored amorphous powder with the empirical formula C₈₀H₁₀₆C₁₂N₁₁O₂₇P·xHCl (where x=1 to 3). In a particular embodiment, telavancin dihydrochloride is used.

Telavancin and telavancin hydrochloride salts can be prepared by methods and processes known in the art. See, for example, U.S. Pat. Nos. 6,635,618 B2; 6,872,701 B2; 6,887,976 B2; 6,979,723 B2; 7,015,305 B2; 7,015,307 B2; 7,074,890 B2; 7,160,984 B2; 7,208,471 B2; 7,301,004 B2; 7,375,181 B2; 7,468,420 B2; 7,531,623 B2; 7,858,583 B2; 8,003,755 B2; and 8,093,354 B2.

### Telavancin Pharmaceutical Composition

Telavancin may be employed as a pharmaceutical composition comprising telavancin or a pharmaceutically-acceptable salt thereof and at least one pharmaceutical excipient. In one embodiment, the pharmaceutical composition consists of telavancin or a pharmaceutically-acceptable salt thereof and at least one pharmaceutical excipient. For example, telavancin hydrochloride is commercially-available under the trademark VIBATIV® (telavancin) as a sterile, preservative-free, white to slightly colored lyophilized powder containing telavancin hydrochloride, 2-hydroxypropyl-β-cyclodextrin and mannitol. The product is available in single-use 250 mg and 750 mg strength vials. The 250 mg strength vial contains telavancin hydrochloride (equivalent to 250 mg of telavancin as the free base); 2500 mg of 2-hydroxypropyl-β-cyclodextrin (Hydroxypropylbetadex, Ph. Eur.); and 312.5 mg of mannitol. The 750 mg strength vial contains telavancin hydrochloride (equivalent to 750 mg of telavancin as the free base); 7500 mg of 2-hydroxypropyl-β-cyclodextrin (Hydroxypropylbetadex, Ph. Eur.); and 937.5 mg of mannitol. Sodium hydroxide and hydrochloric acid are used in minimal quantities for pH adjustment. When reconstituted with, for example, 5% Dextrose Injection, USP; Sterile Water for Injection, USP; or 0.9% Sodium Chloride Injection, USP, these pharmaceutical compositions form a clear to slightly colored solution with a pH of about 4.5±0.5.

In one embodiment, telavancin or a pharmaceutically-acceptable salt thereof may be the only active ingredient in the pharmaceutical composition, together with at least one pharmaceutical excipient. For example, the pharmaceutical composition may consist essentially of telavancin or a pharmaceutically-acceptable salt thereof and at least one pharmaceutical excipient.

In another embodiment, telavancin or a pharmaceutically-acceptable salt thereof may be the only antibiotic agent in the pharmaceutical composition. For example, the pharmaceutical composition may comprise at least one antibiotic agent, wherein the at least one antibiotic agent is telavancin or a pharmaceutically-acceptable salt thereof, and at least one pharmaceutical excipient.

By way of further illustration, telavancin or a pharmaceutically-acceptable salt thereof can be admixed with conventional pharmaceutical excipients and used in the form of aqueous solutions, tablets, capsules, elixirs, suspensions, syrups, wafers, and the like. Such pharmaceutical compositions generally contain, in certain embodiments, from about 0.1 to about 90% by weight of the active compound, and more generally from about 1 to about 30% by weight of the active compound. The pharmaceutical compositions may contain common pharmaceutical excipients, such as corn starch, gelatin, lactose, dextrose, sucrose, microcrystalline cellulose, kaolin, mannitol, dicalcium phosphate, sodium chloride, alginic acid, croscarmellose, sodium starch glycolate, saline, buffered saline, water, glycerol, ethanol, propylene glycol, polysorbate 80 (Tween-80™), poly(ethylene)glycol 300 and 400 (PEG 300 and 400), PEGylated castor oil (e.g. Cremophor EL), poloxamer 407 and 188, and combinations thereof.

Examples of generally used pharmaceutical excipients include, without limitation: stabilizing agents, solubilizing agents, surfactants, buffers, antioxidants, preservatives, tonicity agents, bulking agents, lubricating agents, emulsifiers, suspending agents, viscosity agents, inert diluents, fillers, disintegrating agents, binding agents, wetting agents, lubricating agents, chelating agents, sweeteners, perfuming agents, flavoring agents, coloring agents, and combinations thereof.

The pharmaceutical compositions containing telavancin or a pharmaceutically-acceptable salt thereof may be formulated, for example, for oral, sublingual, intranasal, intraocular, rectal, transdermal, mucosal, topical or parenteral administration for the treatment or prevention of B. anthracis infection. Parenteral modes of administration include, without limitation, intradermal, subcutaneous (s.c., s.q., sub-Q, Hypo), intramuscular (i.m.), intravenous (i.v.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection or infusion of drug formulations can be used to effect such administration.

A liquid pharmaceutical composition may comprise a suspension or solution of telavancin or a pharmaceutically acceptable salt thereof in at least one suitable liquid pharmaceutical excipient, for example, cation-adjusted Mueller-Hinton broth (CAMHB), ethanol, glycerine, sorbitol, non-aqueous solvent such as dimethyl sulfoxide (DMSO), polyethylene glycol, oils sterile water, or water, and may also include a suspending agent, tonicity agent, preservative, surfactant, wetting agent, flavoring agent, coloring agent, or a combination thereof.

Alternatively, a liquid formulation can be prepared from a reconstitutable powder. For example, a powder containing telavancin or a pharmaceutically-acceptable salt thereof can be reconstituted with water to form a suspension; and a syrup can be prepared from a powder containing active ingredient, sucrose and a sweetener.

A pharmaceutical composition in the form of a tablet may also be employed in the methods disclosed herein, for example for oral administration. Such a tablet may be prepared by incorporation of a therapeutically-effective amount of telavancin or a pharmaceutically-acceptable salt thereof with at least one pharmaceutical excipient. Examples of such excipients include magnesium stearate, sodium carbonate, calcium carbonate, sodium phosphate, calcium phosphate, starch, lactose, sucrose, microcrystalline cellulose and binders, for example, acacia, gelatin, sorbitol, methylcellulose, sodium carboxymethylcellulose, poly-vinylpyrrolidone (Povidone), hydroxypropyl methylcellulose, sucrose, starch and ethylcellulose. Lubricants that can be used include magnesium stearate or other metallic stearates, stearic acid, silicone fluid, talc, waxes, oils and colloidal silica. The tablet can also be provided with a color film coating, or color included as part of the at least one pharmaceutical excipient. In addition, telavancin or a pharmaceutically-acceptable salt thereof can be formulated in a controlled release dosage form as a tablet comprising a hydrophilic or hydrophobic matrix.

A pharmaceutical composition in the form of a capsule can be prepared using routine encapsulation procedures, for example, by incorporation of telavancin or a pharmaceutically-acceptable salt thereof and at least one pharmaceutical excipient into a hard gelatin capsule. Alternatively, a semi-solid matrix of telavancin or a pharmaceutically-acceptable salt thereof and high molecular weight polyethylene glycol can be prepared and filled into a hard gelatin capsule; or a solution of telavancin or a pharmaceutically-acceptable salt thereof in polyethylene glycol or a suspension in edible oil, for example, liquid paraffin or fractionated coconut oil can be prepared and filled into a soft gelatin capsule.

Flavoring agents such as peppermint, oil of wintergreen, cherry flavoring or the like can also be used in the pharmaceutical compositions. Additionally, it may be desirable to add a coloring agent to make the dosage form more attractive in appearance or to help identify the product.

Telavancin or a pharmaceutically-acceptable salt thereof can be formulated for intramuscular, intrathecal, or intravenous administration. A typical composition for intramuscular or intrathecal administration will be a suspension or solution of telavancin or a pharmaceutically-acceptable salt thereof in an oil, for example, arachis oil or sesame oil. A typical composition for intravenous or intrathecal administration will be a sterile isotonic aqueous solution containing, for example, telavancin or a pharmaceutically-acceptable salt thereof and dextrose or sodium chloride, or a mixture of dextrose and sodium chloride. Other examples are lactated Ringer's injection, lactated Ringer's plus dextrose injection, Normosol-M and dextrose, Isolyte E, acylated Ringer's injection, and the like. Optionally, a co-solvent, for example, polyethylene glycol, a chelating agent, for example, ethylenediamine tetraacetic acid, and an anti-oxidant, for example, sodium metabisulphite may be included in the formulation. Alternatively, the solution can be freeze dried or lyophilized and then reconstituted with a suitable solvent just prior to administration.

The telavancin or a pharmaceutically-acceptable salt thereof can be formulated as suppositories. A typical suppository formulation will generally comprise telavancin or a pharmaceutically-acceptable salt thereof together with at least one pharmaceutical excipient such as a binding and/or lubricating agent, such as, for example, gelatin or cocoa butter or other low melting vegetable or synthetic wax or fat.

The telavancin or pharmaceutically-acceptable salt thereof can be formulated as transdermal compositions or transdermal delivery devices (“patches”). Such compositions include, for example, a backing, active compound reservoir, a control membrane, liner and contact adhesive. Such transdermal patches may be used to provide continuous or discontinuous infusion of the telavancin in controlled amounts. Such patches may be constructed for continuous, pulsatile, or on demand delivery of telavancin.

In certain embodiments, the pharmaceutical composition containing telavancin or a pharmaceutically-acceptable salt thereof will further comprise a cyclodextrin compound. By way of illustration, the telavancin, for example, in the form a pharmaceutically acceptable salt, can be admixed with an aqueous cyclodextrin solution to form a pharmaceutical composition. Such pharmaceutical compositions will typically contain from about 1 to about 40 weight percent of the cyclodextrin and an effective amount of the telavancin. In certain embodiments, the cyclodextrin employed in the pharmaceutical composition is hydroxypropyl-β-cyclodextrin or sulfobutyl ether β-cyclodextrin. In certain embodiments, the cyclodextrin is hydroxypropyl-β-cyclodextrin. In certain embodiments, the cyclodextrin will comprise about 1 to 40 weight percent; such as about 2 to 30 weight percent; including about 5 to 15 weight percent, of the composition. In an embodiment, the aqueous cyclodextrin solution further comprises dextrose, e.g., about 5% dextrose.

Methods for the Inhibition, Treatment, and Prevention of B. anthracis

Disclosed are methods for the inhibition of B. anthracis and methods for the prevention or treatment of B. anthracis infection in a subject, such as a human.

The disclosed methods may be performed in vivo, in vitro or ex vivo. The in vitro methods are exemplified, but not limited to, methods performed in a laboratory setting, such as in a cell culture, as well as methods performed on inert objects such as laboratory or hospital equipment and devices, surfaces such as countertops and bench tops. The ex vivo methods are exemplified, but not limited to, methods performed on the surface of the human body, such as on the hands.

In each of the disclosed methods, the telavancin, or a pharmaceutically-acceptable salt thereof, may be (a) administered alone, i.e., with no other active ingredient or with no other antibiotic agent or (b) administered in combination with one or more other antibiotic agents or one or more other active ingredients for the inhibition of B. anthracis, prevention of B. anthracis infection, or treatment of B. anthracis infection.

Disclosed are methods of inhibiting B. anthracis bacteria, in vitro, in vivo or ex vivo, comprising contacting B. anthracis with telavancin or a pharmaceutically-acceptable salt thereof in an amount sufficient to inhibit B. anthracis bacteria. Also disclosed are methods of inhibiting the growth of B. anthracis bacteria, in vitro, in vivo or ex vivo, comprising contacting B. anthracis with telavancin or a pharmaceutically-acceptable salt thereof in an amount sufficient to inhibit the growth of B. anthracis bacteria. B. anthracis may be in the form of a vegetative cell, a spore or a mixture of both. The telavancin may be in the form of a pharmaceutical composition comprising telavancin or a pharmaceutically-acceptable salt thereof and at least one pharmaceutical excipient.

Disclosed are methods of inhibiting activation of a B. anthracis spore, in vitro, in vivo or ex vivo, comprising contacting a B. anthracis spore with telavancin or a pharmaceutically-acceptable salt thereof in an amount sufficient to inhibit activation of a B. anthracis spore. Also disclosed are methods of inhibiting germination of a B. anthracis spore, in vitro, in vivo or ex vivo, comprising contacting a B. anthracis spore with telavancin or a pharmaceutically-acceptable salt thereof in an amount sufficient to inhibit germination of a B. anthracis spore. The telavancin may be in the form of a pharmaceutical composition comprising telavancin or a pharmaceutically-acceptable salt thereof and at least one pharmaceutical excipient.

Disclosed are methods of inhibiting outgrowth of a B. anthracis spore, in vitro, in vivo or ex vivo, comprising contacting a B. anthracis spore with telavancin or a pharmaceutically-acceptable salt thereof in an amount sufficient to inhibit outgrowth of a B. anthracis spore. Also disclosed are methods of inhibiting growth of a vegetative form of B. anthracis, in vitro, in vivo and/or ex vivo, comprising contacting a vegetative form of B. anthracis with telavancin or a pharmaceutically-acceptable salt thereof in an amount sufficient to inhibit a vegetative form of B. anthracis. The telavancin may be in the form of a pharmaceutical composition comprising telavancin or a pharmaceutically acceptable salt thereof and at least one pharmaceutical excipient.

Disclosed are methods of treating a B. anthracis infection in a subject, comprising administering a therapeutically effective amount of telavancin or a pharmaceutically-acceptable salt thereof to a subject having a B. anthracis infection. B. anthracis may be in the form of a vegetative cell, a spore, or a mixture of both. The telavancin may be administered in the form of a pharmaceutical composition comprising telavancin or a pharmaceutically-acceptable salt thereof and at least one pharmaceutical excipient. The telavancin or a pharmaceutically-acceptable salt thereof may be administered to the subject within 48 hours of infection, within 36 hours of infection, within 24 hours of infection, within 18 hours of infection, within 12 hours of infection, within 6 hours of infection, within 3 hours of infection, or within 1 hour of infection. In another embodiment, the telavancin or a pharmaceutically-acceptable salt thereof may be administered to the subject after detection of the infection.

Also disclosed are methods of treating a B. anthracis infection in a subject, comprising administering a therapeutically effective amount of telavancin or a pharmaceutically-acceptable salt thereof to a subject having a B. anthracis infection, wherein said treatment inhibits activation of a B. anthracis spore. The telavancin may be administered in the form of a pharmaceutical composition comprising telavancin or a pharmaceutically-acceptable salt thereof and at least one pharmaceutical excipient. The telavancin or a pharmaceutically-acceptable salt thereof may be administered to the subject within 48 hours of infection, within 36 hours of infection, within 24 hours of infection, within 18 hours of infection, within 12 hours of infection, within 6 hours of infection, within 3 hours of infection, or within 1 hour of infection. In another embodiment, the telavancin or a pharmaceutically-acceptable salt thereof may be administered to the subject after detection of the infection.

Also disclosed are methods of treating a B. anthracis infection in a subject, comprising administering a therapeutically effective amount of telavancin or a pharmaceutically-acceptable salt thereof to a subject having a B. anthracis infection, wherein said treatment inhibits germination of a B. anthracis spore. The telavancin may be administered in the form of a pharmaceutical composition comprising telavancin or a pharmaceutically-acceptable salt thereof and at least one pharmaceutical excipient. The telavancin or a pharmaceutically-acceptable salt thereof may be administered to the subject within 48 hours of infection, within 36 hours of infection, within 24 hours of infection, within 18 hours of infection, within 12 hours of infection, within 6 hours of infection, within 3 hours of infection, or within 1 hour of infection. In another embodiment, the telavancin or a pharmaceutically-acceptable salt thereof may be administered to the subject after detection of the infection.

Also disclosed are methods of treating a B. anthracis infection in a subject, comprising administering a therapeutically effective amount of telavancin or a pharmaceutically-acceptable salt thereof to a subject having a B. anthracis infection, wherein said treatment inhibits outgrowth of a B. anthracis spore. The telavancin may be administered in the form of a pharmaceutical composition comprising telavancin or a pharmaceutically-acceptable salt thereof and at least one pharmaceutical excipient. The telavancin may be administered to the subject within 48 hours of infection, within 36 hours of infection, within 24 hours of infection, within 18 hours of infection, within 12 hours of infection, within 6 hours of infection, within 3 hours of infection, or within 1 hour of infection. In another embodiment, the telavancin or a pharmaceutically-acceptable salt thereof may be administered to the subject after detection of the infection.

Also disclosed are methods of treating a B. anthracis infection in a subject, comprising administering a therapeutically effective amount of telavancin or a pharmaceutically-acceptable salt thereof to a subject having a B. anthracis infection, wherein said treatment inhibits growth of a vegetative form of B. anthracis. The telavancin may be administered in the form of a pharmaceutical composition comprising telavancin or a pharmaceutically-acceptable salt thereof and at least one pharmaceutical excipient. The telavancin or a pharmaceutically-acceptable salt thereof may be administered to the subject within 48 hours of infection, within 36 hours of infection, within 24 hours of infection, within 18 hours of infection, within 12 hours of infection, within 6 hours of infection, within 3 hours of infection, or within 1 hour of infection. In another embodiment, the telavancin or a pharmaceutically-acceptable salt thereof may be administered to the subject after detection of the infection.

In the disclosed methods directed to treating a B. anthracis infection, the telavancin or a pharmaceutically-acceptable salt thereof is administered as quickly as possible following exposure to B. anthracis. The telavancin or a pharmaceutically-acceptable salt thereof may be administered to a subject exposed to B. anthracis within 15, 30, 45, 60, 90, or 120 minutes, or within 3, 6, 9, 12, 15, 18, 21, 24, 36, 48, 60 or 72 hours, or within 4, 5, 6, 7, 8, 9, 10, 11, 12, 13, 14, 15, 16, 17, 18, 19, 20, 21, 22, 23, 24, 25, 30, 35, 40, 45, 50, 55, or 60 days, of exposure.

Under some circumstances, the time at which the subject was exposed to B. anthracis cannot be determined, and infection by B. anthracis is only diagnosed upon the onset of clinical symptoms. Under such circumstances, the telavancin or a pharmaceutically-acceptable salt thereof may be administered to a subject within 15, 30, 45, 60, 90, or 120 minutes, or within 3, 6, 9, 12, 15, 18, 21, 24, 36, 48, 60 or 72 hours, or within 4, 5, 6, 7, 8, 9, 10, 11, 12, 13, 14, 15, 16, 17, 18, 19, 20, 21, 22, 23, 24, 25, 30, 35, 40, 45, 50, 55, or 60 days, of the diagnosis of B. anthracis infection.

Also disclosed are methods of preventing a B. anthracis infection in a subject, comprising administering to a subject at risk of exposure to B. anthracis an amount of telavancin or a pharmaceutically-acceptable salt thereof sufficient to prevent B. anthracis infection. The telavancin may be administered in the form of a pharmaceutical composition comprising telavancin or a pharmaceutically-acceptable salt thereof and at least one pharmaceutical excipient. The telavancin or a pharmaceutically-acceptable salt thereof may be administered to the subject less than 48 hours, less than 36 hours, less than 24 hours, less than 18 hours, less than 12 hours or less than 6 hours before risk of exposure to B. anthracis. The exposure to B. anthracis infection may be a cutaneous exposure, exposure by ingestion, exposure by inhalation, or exposure by injection. The duration of prevention of infection may be at least 15 days, 30 days, 45 days or 60 days. In one embodiment, the subject has not previously been exposed to B. anthracis.

Also disclosed are methods for inhibiting colonization of a subject by B. anthracis, comprising administering to a subject at risk of exposure to B. anthracis an amount of telavancin or a pharmaceutically-acceptable salt thereof sufficient to inhibit colonization of a subject by B. anthracis. The telavancin may be administered in the form of a pharmaceutical composition comprising telavancin or a pharmaceutically-acceptable salt thereof and at least one pharmaceutical excipient. The telavancin may be administered to the subject less than 48 hours, less than 36 hours, less than 24 hours, less than 18 hours, less than 12 hours or less than 6 hours before risk of exposure to B. anthracis. The exposure to B. anthracis infection may be a cutaneous exposure, exposure by ingestion, exposure by inhalation or exposure by injection. The duration of prevention of infection may be at least 15 days, 30 days, 45 days or 60 days. In one embodiment, the subject has not previously been exposed to B. anthracis.

In the methods of the present invention directed to preventing a B. anthracis infection and inhibiting colonization by B. anthracis, the telavancin or a pharmaceutically-acceptable salt thereof is administered to the subject less than about 60, 50, 40, 30, 25, 20, 15, 12, 10, 9, 8, 7, 6, 5, 4 or 3 days prior to the risk of exposure to B. anthracis, or less than about 60, 48, 36, 24, 18, 12, 8, 10, 6, 4, 2 or 1 hour prior to the risk of exposure to B. anthracis.

### Preparation and Administration of the Dose

Suitable frequencies for contacting the bacteria with the telavancin or a pharmaceutically-acceptable salt thereof or administering telavancin or a pharmaceutically-acceptable salt thereof to a subject, may vary based on whether administration is for the purposes of inhibition, treatment, or prevention. Administration frequencies for the treatment of a subject having a B. anthracis infection or for prevention of B. anthracis infection include 4, 3, 2 or once daily, every other day, every third day, every fourth day, every fifth day, every sixth day, once weekly, every eight days, every nine days, every ten days, bi-weekly, monthly and bimonthly, and less frequent doses including a single dose.

The therapeutically effective amount of telavancin or a pharmaceutically-acceptable salt thereof varies depending upon, for instance, the physical characteristics of the subject, the severity of the subject's symptoms, the period of time since infection, the formulation and the means used to administer the drug. However, a therapeutically effective amount of telavancin (free base equivalents) may be between about 0.5 mg/kg body weight to 500 mg/kg body weight, from 1 to 100 mg/kg, from 3 to 50 mg/kg, 3 to 30 mg/kg or 3 to 15 mg/kg, regardless of the formulation. In other embodiments, a therapeutically effective amount of telavancin (free base equivalents) is about 0.5, 1, 3, 3.8, 5, 5.6, 10, 15, 20, 25, 30, 35, 40, 45 or 50 mg/kg body weight, regardless of the formulation. In some situations, a dose less than 0.5 mg/kg body weight may be effective.

Accordingly, in one embodiment, for example when administered by IV infusion, the amount of telavancin (free base equivalents) administered is 10 mg/kg body weight once daily. In another embodiment of the invention, the amount of telavancin (free base equivalents) administered is 7.5 mg/kg body weight once daily. In yet another embodiment of the invention, the amount of telavancin (free base equivalents) administered is from 10 to 15 mg/kg body weight once every 48 hours.

The amount of telavancin sufficient to inhibit the growth of B. anthracis bacteria will also vary depending on the environment in which the bacteria is contacted with the telavancin, and the form of the bacteria (e.g., vegetative cell or spore). However, in general the amount of telavancin sufficient to inhibit the growth of B. anthracis bacteria is from 0.001 to 100 μg/ml, from 0.01 to 10 μg/ml, or from 0.01 to 1 μg/ml.

Telavancin or a pharmaceutically-acceptable salt thereof may be administered to a subject by reconstituting a pharmaceutical composition and then further diluting the reconstituted mixture to form a dilute solution suitable for intravenous administration. For example, a 250 mg vial of VIBATIV® (telavancin) can be reconstituted with 15 mL of 5% Dextrose Injection; Sterile Water for Injection; or 0.9% Sodium Chloride Injection. The resultant solution has a concentration of 15 mg/mL and total volume of about 17.0 mL.

Similarly, a 750 mg vial of VIBATIV® (telavancin) can be reconstituted with 45 mL of 5% Dextrose Injection; Sterile Water for Injection; or 0.9% Sodium Chloride Injection. The resultant solution has a concentration of 15 mg/mL and total volume of about 50.0 mL.

Once the telavancin drug product has been reconstituted (e.g., to 15 mg of telavancin (free base equivalents) per mL), the following formula can be used to calculate the volume of reconstituted solution required to prepare a dose:

$$Volume of reconstituted solution ( mL ) = \frac{Telavancin dose ( mg )}{Telavancin concentration ( mg / mL )}$$

For example, a 750 mg dose of telavancin would require 50 mL of reconstituted solution having a telavancin concentration of 15 mg/mL.

For doses of 150 to 750 mg, the appropriate volume of reconstituted solution is typically further diluted with 100 to 250 mL of an infusion solution prior to infusion. Alternatively, doses can be further diluted using a volume that results in a final concentration of 0.6 to 8 mg/mL. Representative infusion solutions include, for example, 5% Dextrose Injection; 0.9% Sodium Chloride Injection; Lactated Ringer's Injection; and the like. The dosing solution may be administered to the subject by intravenous infusion over a period of about 60 minutes, including about 45 to about 75 minutes.

The disclosed methods encompass methods in which telavancin, or a pharmaceutically acceptable salt thereof, is the only active ingredient administered to a subject to treat or prevent a B. anthracis infection. However, it is contemplated that the methods also encompass combination therapy in which telavancin, or a pharmaceutically-acceptable salt thereof, is administered in combination with one or more other active ingredient(s) to treat or prevent a B. anthracis infection. The one or more other active ingredient(s) may be contained in the same pharmaceutical composition as the telavancin or a pharmaceutically-acceptable salt thereof. Alternatively, the one or more other active ingredient(s) may be contained in a separate pharmaceutical composition and may therefore be administered separately, for example before, after, or concurrently with administration of the telavancin or a pharmaceutically-acceptable salt thereof.

The disclosed methods encompass methods in which telavancin, or a pharmaceutically acceptable salt thereof, is the only antibiotic agent administered to a subject to treat or prevent a B. anthracis infection. However, it is contemplated that the methods also encompass combination therapy in which telavancin, or a pharmaceutically-acceptable salt thereof, is administered in combination with one or more other antibiotic agent(s) to treat or prevent a B. anthracis infection. The one or more other antibiotic agent(s) may be contained in the same pharmaceutical composition as the telavancin, or a pharmaceutically-acceptable salt thereof. Alternatively, the one or more other antibiotic agent(s) may be contained in a separate pharmaceutical composition and may therefore be administered separately, for example before, after, or concurrently with administration of the telavancin or a pharmaceutically-acceptable salt thereof.

Other antibiotic agents include, but are not limited to, fluoroquinolones (including ciprofloxacin), tetracyclines (including doxycycline), glycopeptides (including vancomycin and oritavancin), macrolides (including erythromycin, cethromycin, azithromycin and clarithromycin), β-lactams (including penicillin, imipenem and ampicillin), ansamycins (including rifampin), phenicols (including chloramphenicol), streptogramins (including quinupristin-dalfopristin), aminoglycosides (including gentamicin), oxazolidinones (including linezolid), tetracyclines, glycylglycines (including tigecycline), cyclic lipopeptides (including daptomycin) and lincosamines (including clindamycin).

### EXAMPLES

The following examples are not meant to be limiting and represent certain embodiments of the present invention.

### Example 1: Evaluation of VIBATIV® (Telavancin) Against a Panel of B. anthracis Strains as Measured by Broth Microdilution

Broth microdilution minimum inhibitory concentrations (MICs) were determined for VIBATIV® (telavancin) against the following 17 strains of B. anthracis: Ames 3838, Graves, 46-PY-5, Ames, Kruger B (A0442), Vollum (A0488), WNA, A0318, A0471, ASC 506, ASC 525, 2000032823 (CDC #1), 2002734753 (CDC #2), 2010719149 (CDC #3), 2006200760 (CDC #4), ASC 32, and ASC 149. MICs for comparator antibiotic doxycycline was determined in parallel.

VIBATIV® (telavancin) was dissolved at a concentration of 6.4 mg/ml and diluted to 256 μg/ml in the first column of a 96 well plate (final concentration was 128 μg/ml when diluted 1:2 with culture). Specifically, the lyophilized cake of VIBATIV® (telavancin) contained in a vial was broken up by shaking and tapping the vial prior to adding diluent to maximize surface area. Subsequently, 45 ml sterile 0.85% saline was added to the vial via syringe and the vacuum was allowed to pull the diluent into the vial. The vial was gently rolled between the hands in order to dissolve the powder. The resulting concentration was 15 mg/ml, based on 90% purity of VIBATIV® (telavancin) in powder. The solution was further diluted to 6.4 mg/ml in sterile 0.85% saline for use as working stock for drug plate preparations.

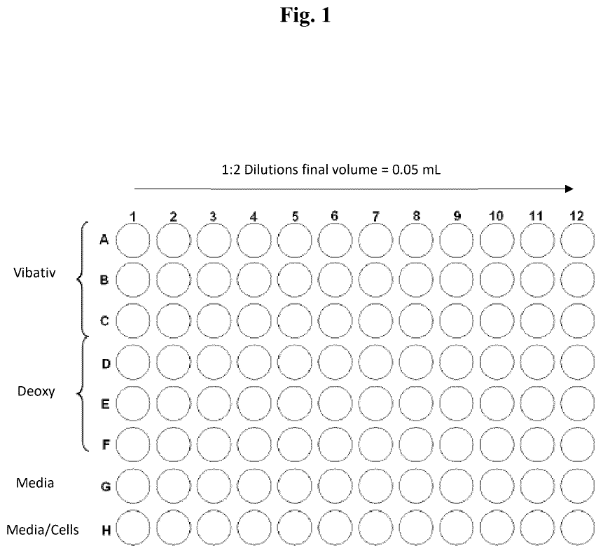

Doxycycline (Sigma D9891) was prepared as a positive test control. The doxycycline was dissolved in DMSO at a concentration of 1.6 mg/ml and diluted to 64 μg/ml in the first column of a 96 well plate (final concentration was 32 μg/ml when diluted 1:2 with culture). As shown in FIG. 1, 96-well round bottom plates with VIBATIV® (telavancin) and doxycycline solutions were prepared.

10 ul glycerol stock was added into 10 ml Tryptic Soy Broth (VWR 90000-378) in a 50 ml conical tube. The tube was incubated on an orbital shaker at 250 rpm at 37° C. for 18 hours. The optical density (OD) of the culture was measured at a wavelength of 600 nm. The results are shown in Table 1. The starter culture was diluted 1:100 into a 50 ml conical tube in 10 ml Tryptic Soy Broth. The tube was incubated on an orbital shaker at 250 rpm at 37° C. for 6 hours. The optical density (OD) of the passed culture was measured at a wavelength of 600 nm. The results are shown in Table 1. The culture was diluted to an OD of 0.1 (approx. 1×10⁸ CFU/ml) and further diluted 1:100 (1×10⁶ CFU/ml) in 15 ml total volume cation-adjusted Mueller-Hinton (caMH) broth. Subsequently, 0.05 ml of the culture was added to each well of the plate, except 0.1 ml of the culture was added to the wells in Column 1 and to row G, as shown in FIG. 1. The plate was incubated at 37° C. for 18 hours. Pellet growth was observed in each of the wells and minimum inhibitory concentration (MIC) was measured as the first well to show no observable growth. The results are shown in Table 1.

TABLE 1

| | O/N | Passed | | |
|---|---|---|---|---|
| Strain | OD | Culture OD | Vibativ | Doxycycline |
| Ames 3838 | 0.99 | 1.02 | <0.0625 | 0.03125 |
| Graves | 1.12 | 1.03 | <0.0625 | 0.03125 |
| 46-PY-5 | 1.10 | 1.11 | <0.0625 | 0.03125 |
| Ames | 1.26 | 0.98 | <0.0625 | 0.03125 |
| Kruger B (A0442) | 1.49 | 1.20 | 0.125 | <0.0156 |
| Vollum (A0488) | 1.28 | 1.02 | <0.0625 | 0.03125 |
| WNA | 1.52 | 1.59 | <0.0625 | 0.03125 |
| A0318 | 1.24 | 1.04 | <0.0625 | 0.03125 |
| A0471 | 1.22 | 1.14 | <0.0625 | 0.03125 |
| Anthrax Strain | 1.36 | 1.05 | <0.0625 | 0.03125 |
| Collection 506 | | | | |
| Anthrax Strain | 1.51 | 1.00 | <0.0625 | 0.03125 |
| Collection 525 | | | | |
| 2000032823 (CDC #1) | 1.00 | 0.83 | <0.0625 | <0.0156 |
| 2002734753 (CDC #2) | 0.82 | 0.64 | 0.125 | 0.03125 |
| 2010719149 (CDC #3) | 1.20 | 1.00 | <0.0625 | 0.03125 |
| 2006200760 (CDC #4) | 0.59 | 0.77 | <0.0625 | <0.0156 |
| ASC 32 | 1.38 | 1.10 | <0.0625 | 0.03125 |
| ASC 149 | 1.02 | 1.03 | <0.0625 | 0.03125 |

VIBATIV ® (telavancin) generally showed very good activity against B. anthracis with MIC at or below 0.125 μg/ml.

### Example 2: Evaluation of Post-Exposure Administration of VIBATIV® (Telavancin) Against Inhalational Anthrax in a Rabbit Model

Demonstration of activity of an antibacterial agent, such as telavancin, in an animal model is of significant impact to the identification of doses and dose regimens that would provide effective therapy in humans because phase II and phase III clinical trials (on anthrax-infected subjects) cannot be conducted for ethical reasons. As such, studies with anthrax-infected animals are critical to approve agents for anthrax treatment (see, e.g., Federal Register, 67 FR 37988-37998 (2002); Guidance for Industry. Anthrax: Developing Drugs for Prophylaxis of Inhalational Anthrax, CDER May 2018). All animal-related procedures for this study were conducted in accordance with an animal use protocol approved by the University of Texas Medical Branch (UTMB) Institutional Animal Care and Use Committee (IACUC).

The basic study design is shown in Table 2. A total of 28 adult New Zealand White (NZW) rabbits were randomized on the basis of gender (50% males and 50% females) into the three experimental groups listed in Table 2. All animals were challenged individually via muzzle-only aerosol with 200 LD₅₀ (2.0×10⁷ CFUs) of purified B. anthracis Ames spores using real-time plethysmography and a muzzle-only aerosol exposure chamber. Treatment commenced upon detection of protective antigen (PA) in sera. Blood samples were collected pre-challenge and at various times post-challenge for determination of bacterial/spore load. Furthermore, serum samples were collected at the various time-points to assess the levels of PA and anti-PA IgG.

Daily clinical observations, which included daily weights, were recorded on the day of challenge, to determine baseline measures/observations, and through the end of the designated in-life period to evaluate the progress of the infection and all clinical outcomes. Temperature was also recorded via implanted data loggers for each animal starting upon placement on the study and continued through the end of the in-life phase of the study. Survival was monitored for approximately 14 days post-challenge, after which time all survivors were euthanized and necropsied, and terminal samples taken. Any animal found to be moribund or in respiratory distress prior to the end of the in-life phase was humanely euthanized and necropsied. Lastly, tissue samples (lung, mediastinal lymph nodes, brain, and spleen) were collected from all animals either at the time of euthanasia or at the end of the in-life period for determination of bacterial/spore load and histopathology (liver, kidneys, and heart were also collected for histopathology). If an animal was found dead, the time-to-death was determined as accurately as possible, and any terminal samples that could be recovered were taken.

TABLE 2
Experimental design

| | | | Spore | | | | |
|---|---|---|---|---|---|---|---|
| | | | Challenge | | | | |
| | Test | Number of | Dose (Ames, | Vibativ | Levofloxacin | | In-Life |
| Group | Article | Rabbits | NR3838) | Dose | Dose | Dose Regimen | Period |
| 1 | Vibativ | 12 | ~200LD₅₀ | 30 mg/kg | 0 | At + PA-ECL, | 14 d |
| | | | | | | IV, BID, 5 d | |
| 2 | Levofloxacin | 12 | | 0 | 12.5 mg/kg | At + PA-ECL, | |
| | | | | | | IV, SID, 5 d | |
| 3 | Saline | 6 | | 0 | 0 | At + PA-ECL, | |
| | | | | | | IV, SID, 5 d | |

A total of 30 NZW rabbits, 50% male and 50% female, were purchased from Covance (Denver, PA). This included 2 extra animals to serve as replacements if needed; however, these extra animals were not needed as replacements and were added to the study's saline-treated group. A venous access port (VAP) was surgically implanted into the external jugular vein of each animal to facilitate both the collection of multiple blood specimens and the I.V. administration of the therapeutics during the study. For this study, the 30 animals were divided into two cohorts, which arrived four weeks apart (the 2 extra animals arrived in the last cohort). The animals were distributed into the three experimental groups within each cohort according to Table 3. As noted in Table 3, each cohort had an equal number of males and females. After arrival, the animals acclimated for at least 72 hours during which time each was given a thorough physical examination by a veterinarian.

TABLE 3
Group/cohort animal distribution

| | Group 1 | Group 2 | Group 3 | Total |
|---|---|---|---|---|
| Cohort 1 | 6 | 6 | 2 | 14 |
| | (3M, 3F) | (3M, 3F) | (1M, 1F) | (7M, 7F) |
| Cohort 2 | 6 | 6 | 4 | 16 |
| | (3M, 3F) | (3M, 3F) | (2M, 2F) | (8M, 8F) |
| Total | 12 | 12 | 6 | 30 |
| | (6M, 6F) | (6M, 6F) | (3M, 3F) | (15M, 15F) |

Following acclimation, each animal was implanted intraperitoneally with a temperature data logger for the purpose of recording the animals' temperature for the entire in-life period, after which the data were downloaded and analyzed. After 10-14 days of recovery, the animals were transferred to the facility where the aerosol challenge and all subsequent experimental procedures were performed.

Rabbits were individually challenged by aerosol with 200 LD₅₀ of purified B. anthracis Ames NR-3838 spores using an automated aerosol control platform (Biaera Technologies, Hagerstown, MD), fitted with a head-only aerosol exposure chamber, a 6-jet Collison nebulizer, and BioSampler (SKC, Eighty Four, PA). A dedicated computer was used for setting aerosol-related run parameters, as well as for monitoring humidity, pressure, and air flow rates during each exposure run. Real-time plethysmography was performed on each rabbit using a pair of elastic band sensors (DSI, New Brighton, MN) placed around the animal's thorax and abdomen, which were then calibrated with a pneumotach fitted to the face of each animal. After calibration, the rabbit's head was inserted into the aerosol head-only exposure chamber. The target dose of spores (D_{P}) for aerosol deposition in the lungs was 2.0×10⁷ cfu. The 6-jet Collison nebulizer used to generate the aerosols ideally yields a S_{f} of 1.0-2.0×10⁻⁶ for B. anthracis spores. The nebulizer concentration to deliver this dose was calculated using standard algorithms (Roy CJ, LML Pitt. 2005. Infectious disease aerobiology: Aerosol challenge methods. In: Biodefense: Research Methodology and Animal Models, Swearengen JL (Ed.). CRC Press, Boca Raton, FL), combined with a standard volume of air to deliver the target challenge dose of spores. Aerosol samples were collected continuously for confirming the challenge dose of spores for each animal by serial dilution and plating on blood agar plates. The duration of aerosol delivery was based on the respiration rate of each animal, plotted on the computer screen, and controlled by the aerosol system computer (Biaera Technologies, Hagerstown, MD).

Upon receipt, VIBATIV® (telavancin) was stored at 2-8° C., as directed by the manufacturer, with temperature monitoring. Levofloxacin, upon receipt, was stored at room temperature with temperature monitoring and protected from light. For therapeutic treatment of Group 1, the appropriate amount of VIBATIV® (telavancin) for each animal was freshly prepared daily by dissolving in sterile, pyrogen-free water to a concentration of 25 mg/ml and administered IV via the implanted VAP at 30 mg/kg BID (every 12 h) for 5 days (Table 4a, 4b). Group 2 (positive controls) received levofloxacin IV, via the VAP, at a concentration of 12.5 mg/kg once a day for 5 days. The appropriate dilutions of levofloxacin were prepared fresh daily using sterile, pyrogen-free water. Lastly, Group 3 (negative control) received saline once a day for 5 days administered IV via the VAP. All treatments commenced upon detection of protective antigen (PA) in the animals' sera (within 3 hours after blood collection).

The animals were not treated on an individual basis for the duration of the entire treatment period. The first dose administrations were prepared on an individual animal basis; however, all subsequent daily doses were prepared for treatment groups and delivered during the morning of each dosing day. The second treatment dose occurred in the AM of the next calendar day regardless of the time of initial treatment, and all subsequent treatments occurred in the AM. For example, if Rabbit X was PA positive and initial treatment occurred at 03:00 hours on 27 Aug. 2020, the next treatment was in the AM (~08:00-10:00) of 28 Aug. 2020. Rabbit X was treated during the AM for the rest of the study.

TABLE 4a
Therapeutic Dosing and Bleeding Schedule, Days −7 to 2.5

| | Days Post-Challenge | | | | | | | | | | | |
|---|---|---|---|---|---|---|---|---|---|---|---|---|
| | | | Challenge | | | | | | | | | |
| | n | −7 | 0 | 0.5 | 0.75 | 1 | 1.25 | 1.5 | 1.75 | 2 | 2.25 | 2.5 |
| | Hours Post-Challenge | | | | | | | | | | | |
| | n | −168 | 0 | 12 | 18 | 24 | 30 | 36 | 42 | 48 | 54 | 60 |
| Vibativ | 12 | ◯ § # | — | ◯ § | § | Vibativ | §* | Vibativ | §* | Vibativ | §* | Vibativ |
| 30 mg/kg | | | | | | ◯ §* | | §* | | ◯ §* | | §* |
| Levofloxacin | 12 | ◯ § # | — | ◯ § | § | Levo | §* | §* | §* | Levo | §* | §* |
| 12.5 mg/kg | | | | | | ◯ §* | | | | ◯ §* | | |
| Control | 6 | ◯ § # | — | ◯ § | ◯ | Saline | §* | §* | §* | Saline | §* | §* |
| (Saline) | | | | | | ◯ §* | | | | ◯ §* | | |

TABLE 4b
Therapeutic Dosing and Bleeding Schedule, Days 2.75 to 14

| | Days Post-Challenge | | | | | | | | | | |
|---|---|---|---|---|---|---|---|---|---|---|---|
| | n | 2.75 | 3 | 3.5 | 4 | 4.5 | 5 | 5.5 | 7 | 10 | 14 |
| | Hours Post-Challenge | | | | | | | | | | |
| | n | 66 | 72 | 84 | 96 | 108 | 120 | 132 | 168 | 240 | 336 |
| Vibativ | 12 | §* | Vibativ 0 | Vibativ | Vibativ | Vibativ | Vibativ | Vibativ | ◯ # | ◯ # | ◯ # |
| 30 mg/kg | | | ◯ §* # | | ◯ | | ◯ | | | | |
| Levofloxacin | 12 | §* | Levo | | Levo | | Levo | | ◯ # | ◯ # | ◯ # |
| 12.5 mg/kg | | | ◯ §* # | | ◯ | | ◯ | | | | |
| Control | 6 | §* | Saline | | Saline | | Saline | | ◯ # | ◯ # | ◯ # |
| (Saline) | | | ◯ §* # | | ◯ | | ◯ | | | | |

Blood specimens were collected from each animal, via the VAP, prior to challenge and at specified times post-challenge (Tables 4a, 4b; o=Blood collection for bacteremia; §=Blood collection for antigenemia; #=Blood collection for Anti-PA IgG assessment; *=If necessary; Day 0=aerosol challenge). In the event of catheter failure (e.g., loss of patency), blood specimens were collected from the central ear artery. This alternate method of blood collection was proposed because B. anthracis in the blood of bacteremic animals has the potential to colonize the VAP (or catheter), thereby contaminating successive samples drawn from the port. The impact of catheter colonization could have resulted in reporting an animal as bacteremia positive when in actuality the bacteria had already been cleared from the blood by therapeutic treatment. Therefore, blood specimens used for the purpose of assessing bacteremia beginning 7 days post-challenge were collected from a peripheral blood vessel. Blood specimens were collected into 0.5 ml blood collection microtubes (Wampole Laboratories, Cranbury, NJ) for quantitative bacterial plate counts at the specified time-points (Table 4). Blood was also collected in 2.0 ml serum separator (1.5 ml) at selected times pre- and post-challenge and the serum used for PA-ECL and anti-PA IgG titration (Table 4). Sera for IgG testing were stored frozen (−20° C.) and samples tested as a batch at the conclusion of the in vivo portion of the study. Approximately 2.0 ml of whole blood was collected from each animal at the collection times specified above for the various assays.

Bacterial concentration in the blood was determined using an automatic serial diluter and plater (easySpiral Dilute; Interscience). Whole blood, diluted in sterile water, was plated onto trypticase soy agar II plates containing 5% sterile sheep blood (TSAII) and incubated at 37° C. for 16-24 hours. Colonies from the plates were then enumerated using an automatic colony counter (Scan 500; Interscience). Bacterial colonies having morphology typical of B. anthracis were subcultured and confirmed as B. anthracis with bacteriophage y.

Bacterial/spore load was also determined in lung, lymph node (mediastinal), brain, and spleen. These tissues were homogenized in sterile water using a Stomacher 80 MicroBiomaster (Seward Ltd), and the homogenate was serially diluted in water and plated onto TSAII plates using the automatic diluter/plater and incubated at 37° C. for 16-24 hours. Colonies from the plates were then enumerated using an automatic colony counter (Scan 500; Interscience), and the bacterial load was presented as cfu per gram of tissue. Bacterial colonies having morphology typical of B. anthracis were subcultured and confirmed as B. anthracis with bacteriophage y.

B. anthracis PA was measured in serum using a rapid PA-ECL screening assay (MesoScale Discovery; Gaithersburg, MD). This assay kit utilizes a detection antibody in combination with specialized 96-well microtiter plates that contain electrodes coated with an anti-PA capture antibody to detect and/or quantify PA. Following processing and assay execution, the amount of light emitted in sample wells is used to directly measure the amount of PA present in the serum based on a recombinant PA standard curve run in parallel. To quantitate the levels of PA in each serum sample, a standard curve (0-100 ng/ml) was analyzed in parallel on each assay day. Test samples were assayed in duplicate. The concentration of each test sample was extrapolated from the standard curve. Values that fell below the lower limits of the standard curve were defined as LLOQ (lower limit of quantitation).

Anti-PA IgG was measured in serum via ECL similar to the PA-ECL screening assay. Biotinylated recombinant PA63 was bound to streptavidin-coated plates (MSD) and used as the capture antigen. Detection was accomplished using SULFO-TAG labeled anti-rabbit antibody and read buffer (MSD). Results were presented as fold-increase in signal relative to the pre-challenge serum samples.

Necropsies were performed by veterinarians on animals that either succumbed to infection or were euthanized. In addition to gross pathology, selected tissue fixation, embedding, sectioning, and staining was performed for microscopic pathology. Histopathology was performed on H&E-stained sections of lung, lymph nodes (mediastinal), brain, spleen, liver, kidneys, and heart.

During the in-life phase of the study, clinical observations were performed/recorded at least twice daily, with more observations being performed during the peak time of infection when the animals showed increased signs of disease. The animals' weights were recorded daily starting upon placement on the study and continuing through the end of the in-life period of the study. Any animal found to be immobile (severe lack of movement in response to stimulation), unable to get to food/water, and/or in respiratory distress (abdominal breathing, open mouth breathing, nasal flaring) was immediately euthanized, and the time of death was recorded. At the time of euthanasia, all terminal samples were also collected. If an animal was found dead, the time of death was determined as accurately as possible, and any terminal samples able to be recovered were taken. Lastly, all survivors were humanely euthanized on the last day of the in-life period, and all terminal samples taken.

A two-factor experiment was conducted using a randomized complete block design (RCB). Blocks (gender) are males and females. For example, one factor was the treatments while the second factor was the time on trial. Male and female rabbits (experimental units within blocks 1 or 2, respectively) were randomized into the indicated treatment groups. Binary outcome variables (e.g., animal death) were analyzed using Kaplan-Meier Survival analyses to compare each of the survival curves. For measurement variables, Analysis of Variance (ANOVA) methods appropriate for a two-factor factorial experiment in an RCB design were used. Overall treatment differences, gender differences and their interaction were tested using pooled inter-animal variability as the error variance, while differences across time, as well as the time by treatment interaction and the post hoc significance of treatment differences at specific time intervals within each sex, were tested using the pooled “animal by treatment” interaction. If means and standard deviations correlated, the response measures were transformed before analysis. Significance levels for directional hypotheses were used and the Tukey-Kramer method helped prevent an undue number of false positives. Since animals were expected to die at different intervals, a series of repeated ANOVA measures was conducted to account for time-related losses among animals on treatment.

During aerosol exposure, the rabbits' respiration was monitored/recorded in real-time using a DSI plethysmography system that was integrated with the Biaera aerosol control platform. For each animal, a target accumulated total volume (ATV) was set to 7,000 ml which was predetermined to give a presented dose (D_{P}) of 2.0×10⁷ spores. The D_{P} denotes the number of spores deposited into the animals' lungs. As soon as the animal inhaled 7,000 ml of air, the aerosol exposure to spores was terminated by the computer. The aerosol exposure times among all animals in the two cohorts ranged from 6 to 24 min. The range of exposure times for all cohorts was due to the animals' varying respiration intensities that resulted from their differing levels of sedation during the aerosol challenge. Nonetheless, despite the various exposure times, all animals were challenged with comparable challenge doses due to the capabilities of the real-time plethysmography system. The cumulative mean D_{P} for the two cohorts was 2.25×10⁷ spores (target was 2.0×10⁷ spores) which is 225 LD₅₀. The overall mean S_{F} was 2.08×10⁻⁶. S_{F}, which conveys the efficiency of each aerosol spray, is a unitless parameter defined as the spore concentration of the aerosol (C_{AERO}) divided by the spore concentration of the nebulizer suspension (C_{NEB}). A mean S_{F} of 2.08×10⁻⁶ indicates an overall high efficiency of aerosolization.

Treatments were prepared and administered upon detection of PA in the animals' sera. Table 5 provides the serum PA concentration for each animal and shows the time-point at which PA was first detected. PA was detected as early as 18 hours post-challenge and as late as 30 hours post-challenge, while the majority of animals were positive for PA at 18 to 24 hours post-challenge (PC). The concentration of PA in the sera ranged from approximately 50 to 2,000 μg/ml. Value reported as <LLOQ (lower limit of quantitation) denotes measurements that were below the standard curve.

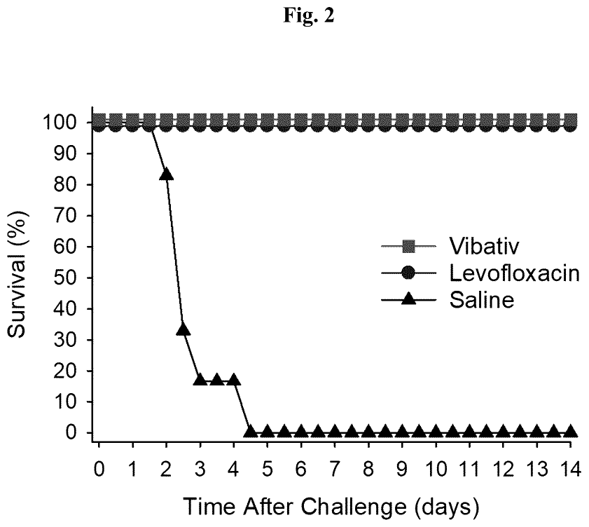

FIG. 2 shows the survival results for the treatment groups over the 14-day post-challenge period. The VIBATIV® (telavancin)-treated group exhibited 100% survival after challenge which was significantly (p<0.001) greater than that of the saline-treated group which showed no survival. As expected, the animals administered saline succumbed to infection 2 to 4 days post-challenge. The group treated with the humanized dose of levofloxacin also showed 100% survival. These results show that VIBATIV® (telavancin) was completely protective in this model of inhalation anthrax infection.

TABLE 5
B. anthracis antigenemia levels

| Group | Animal ID# | −7 days | 12 h PC | 18 h PC | 24 h PC | 30 h PC |
|---|---|---|---|---|---|---|
| Vibativ | 8629 | 0 | <LLOQ | 960 | | |
| | 8630 | 0 | 0 | 597 | | |
| | 8633 | 0 | 0 | 824 | | |
| | 8637 | 0 | 0 | 371 | | |
| | 8639 | 0 | 0 | 796 | | |
| | 8641 | 0 | 0 | 0 | 348 | |
| | 8643 | 0 | <LLOQ | 1,781 | | |
| | 8646 | 0 | 0 | <LLOQ | 2,039 | |
| | 8647 | 0 | 0 | <LLOQ | 639 | |
| | 8651 | 0 | 0 | 418 | | |
| | 8653 | 0 | 0 | <LLOQ | 841 | |
| | 8656 | 0 | 0 | 377 | | |
| Levofloxacin | 8628 | 0 | <LLOQ | 1,052 | | |
| | 8632 | 0 | 0 | 1,128 | | |
| | 8634 | 0 | 0 | 58.4 | | |
| | 8635 | 0 | 0 | <LLOQ | 468 | |
| | 8638 | 0 | 0 | 146 | | |
| | 8640 | 0 | 0 | 154 | | |
| | 8642 | 0 | 0 | <LLOQ | 514 | |
| | 8644 | 0 | 0 | 0 | <LLOQ | 258 |
| | 8649 | 0 | 0 | <LLOQ | 1,189 | |
| | 8652 | 0 | <LLOQ | 1,229 | | |
| | 8654 | 0 | <LLOQ | 146 | | |
| | 8657 | 0 | 0 | <LLOQ | 269 | |
| Saline | 8631 | 0 | 0 | 509 | | |
| | 8636 | 0 | 0 | 0 | <LLOQ | 1,007 |
| | 8645 | 0 | 0 | 922 | | |
| | 8648 | 0 | <LLOQ | 389 | | |
| | 8650 | 0 | 0 | 746 | | |
| | 8655 | 0 | 0 | 0 | 164 | |

The antibiotic-treated groups (VIBATIV® (telavancin) and levofloxacin) exhibited comparable temperature responses after challenge. Specifically, animals in both groups had febrile responses at approximately Day 1 post-challenge that peaked at nearly 41° C. before Day 2. By Day 2, the average core body temperatures for the antibiotic-treated groups returned to baseline, most likely due to treatment which began at approximately 24 hours post-challenge. Both antibiotic-treated groups appeared to have minor secondary febrile responses from 7-9 days post-challenge which was after treatment was ended (Day 6), but these temperature elevations subsided by Day 10 to 11. The average temperature of the animals treated with saline also began to rise by Day 1, and it remained elevated until hours before the animals succumbed to infection. These results show that post-exposure treatment with VIBATIV® (telavancin), by limiting bacterial progression, was effective at reducing and/or preventing the febrile response consistently reported with anthrax infection.

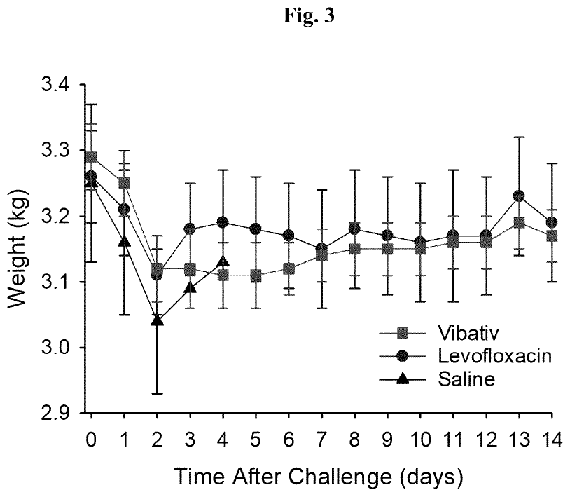

FIG. 3 shows the average weight (with standard error bars) for each treatment group during the post-challenge period. The average weights for all treatment groups began to drop after challenge, and this reduction in weight continued for approximately 2 days post-challenge. Thereafter, the average weights for the antibiotic groups began to either stabilize and/or rebound, but the resurgence appeared to take longer for the VIBATIV® (telavancin)-treated group compared to the levofloxacin-treated group. The average weight of the saline-treated group appeared to have increased after Day 2 as well; however, this increase was simply due to removal of animals that had succumbed to infection.

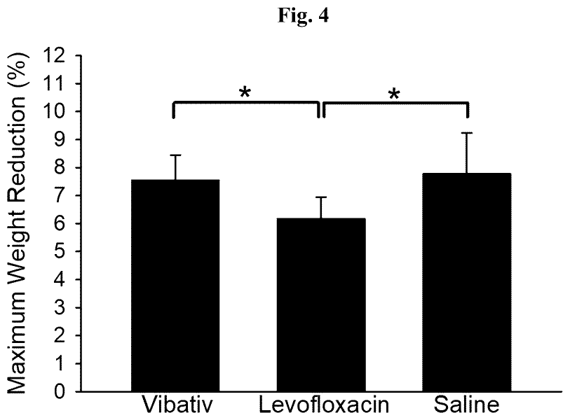

The maximum percent weight loss of the VIBATIV® (telavancin)-treated group was significantly (p<0.05) higher than that of the levofloxacin-treated group (FIG. 4), but this was due in large part to the differences among the female animals in the treatment groups. The maximum percent weight reductions among the male animals in the treatment groups were more comparable. Altogether, these data suggest that the treatment dose/regimen for VIBATIV® (telavancin) used in this study was possibly associated with slight weight reduction not seen with levofloxacin treatment.

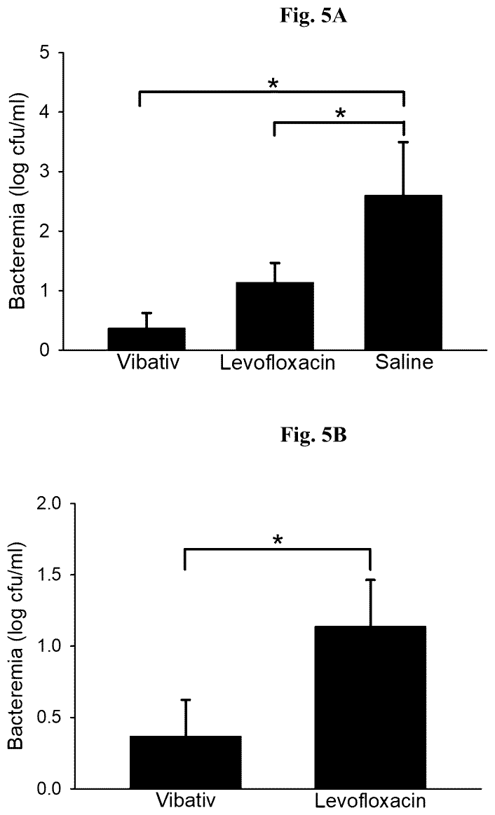

Table 6 indicates the B. anthracis bacterial load in the blood for each animal in the three treatment groups. Table 7 gives the B. anthracis bacterial load in lung tissue collected from each animal in the three treatment groups at time of euthanasia due to reaching humane or scientific endpoints. Bacteremia was detected as early as 24 hours post-challenge, at which time the average level of bacteremia for the antibiotic-treated groups was significantly (p<0.05) lower than that of the saline-treated group (primarily due to differences in female animals) (FIG. 5a). Approximately half the animals in both the VIBATIV® (telavancin)- and levofloxacin-treated groups received their initial dose of antibiotics by 24 hours post-challenge, but interestingly, only 2 out of 12 animals treated with VIBATIV® (telavancin) were positive

TABLE 6
B. anthracis bacteremia levels

| | Animal | | 12 h | 24 h | 48 h | 72 h | 96 h | 120 h | 7 days | 10 days | 14 days |
|---|---|---|---|---|---|---|---|---|---|---|---|
| | ID# | −7 days | PC | PC | PC | PC | PC | PC | PC | PC | PC |
| Vibativ | 8629 | 0 | 0 | 0 | 0 | 0 | 0 | 0 | 0 | 0 | 0 |
| | 8630 | 0 | 0 | 0 | 0 | 0 | 0 | 0 | 0 | 0 | 0 |
| | 8633 | 0 | 0 | 0 | 0 | 0 | 0 | 0 | 0 | 0 | 0 |
| | 8637 | 0 | 0 | 0 | 0 | 0 | 0 | 0 | 0 | 0 | 0 |
| | 8639 | 0 | 0 | 0 | 0 | 0 | 0 | 0 | 0 | 0 | 0 |
| | 8641 | 0 | 0 | 0 | 0 | 0 | 0 | 0 | 0 | 0 | 0 |
| | 8643 | 0 | 0 | 0 | 0 | 0 | 0 | 0 | 0 | 0 | 0 |
| | 8646 | 0 | 0 | 3.90E+02 | 0 | 0 | 0 | 0 | 0 | 0 | 0 |
| | 8647 | 0 | 0 | 0 | 0 | 0 | 0 | 0 | 0 | 0 | 0 |
| | 8651 | 0 | 0 | 0 | 0 | 0 | 0 | 0 | 0 | 0 | 0 |
| | 8653 | 0 | 0 | 7.00E+01 | 0 | 0 | 0 | 0 | 0 | 0 | 0 |
| | 8656 | 0 | 0 | 0 | 0 | 0 | 0 | 0 | 0 | 0 | 0 |
| Levo | 8628 | 0 | 0 | 1.00E+01 | 0 | 0 | 0 | 0 | 0 | 0 | 0 |
| | 8632 | 0 | 0 | 7.00E+01 | 0 | 0 | 0 | 0 | 0 | 0 | 0 |
| | 8634 | 0 | 0 | 0 | 0 | 0 | 0 | 0 | 0 | 0 | 0 |
| | 8635 | 0 | 0 | 1.00E+01 | 0 | 0 | 0 | 0 | 0 | 0 | 0 |
| | 8638 | 0 | 0 | 0 | 0 | 0 | 0 | 0 | 0 | 0 | 0 |
| | 8640 | 0 | 0 | 0 | 0 | 0 | 0 | 0 | 0 | 0 | 0 |
| | 8642 | 0 | 0 | 2.00E+01 | 0 | 0 | 0 | 0 | 0 | 0 | 0 |
| | 8644 | 0 | 0 | 0 | 0 | 0 | 0 | 0 | 0 | 0 | 0 |
| | 8649 | 0 | 0 | 4.42E+03 | 0 | 0 | 0 | 0 | 0 | 0 | 0 |
| | 8652 | 0 | 0 | 3.60E+02 | 0 | 0 | 0 | 0 | 0 | 0 | 0 |
| | 8654 | 0 | 0 | 2.00E+01 | 0 | 0 | 0 | 0 | 0 | 0 | 0 |
| | 8657 | 0 | 0 | 1.00E+01 | 0 | 0 | 0 | 0 | 0 | 0 | 0 |
| Saline | 8631 | 0 | 0 | 1.90E+05 | 4.20E+04 | | | | | | |
| | 8636 | 0 | 0 | 0 | 9.55E+02 | 8.50E+03 | 1.76E+06 | | | | |
| | 8645 | 0 | 0 | 6.71E+03 | 4.29E+04 | 5.60E+06 | | | | | |
| | 8648 | 0 | 0 | 2.50E+02 | 8.20E+05 | | | | | | |
| | 8650 | 0 | 0 | 1.16E+04 | 1.45E+06 | | | | | | |
| | 8655 | 0 | 0 | 0 | 1.10E+06 | | | | | | |

TABLE 7
B. anthracis bacterial load in lung tissues

| | | | Time of | | |
|---|---|---|---|---|---|
| | | | tissue | | Average |
| | | | collection | Bacterial | Bacterial |
| | Animal | | (time post- | Load | Load |
| Group | ID# | Tissue | challenge) | (cfu/g) | (cfu/g) |
| Vibativ | 8629 | Lung | 14 days | 1.83E+03 | 1.93E+03 |
| | 8630 | Lung | 14 days | 5.03E+02 | |
| | 8633 | Lung | 14 days | 7.71E+02 | |
| | 8637 | Lung | 14 days | 8.03E+02 | |
| | 8639 | Lung | 14 days | 1.60E+03 | |
| | 8641 | Lung | 14 days | 2.25E+03 | |
| | 8643 | Lung | 14 days | 1.07E+03 | |
| | 8646 | Lung | 14 days | 1.18E+03 | |
| | 8647 | Lung | 14 days | 4.52E+03 | |
| | 8651 | Lung | 14 days | 1.74E+03 | |
| | 8653 | Lung | 14 days | 5.08E+03 | |
| | 8656 | Lung | 14 days | 1.78E+03 | |
| Levofloxacin | 8628 | Lung | 14 days | 2.88E+04 | 6.93E+03 |
| | 8632 | Lung | 14 days | 3.43E+03 | |
| | 8634 | Lung | 14 days | 2.69E+03 | |
| | 8635 | Lung | 14 days | 2.77E+03 | |
| | 8638 | Lung | 14 days | 3.50E+03 | |
| | 8640 | Lung | 14 days | 8.72E+02 | |
| | 8642 | Lung | 14 days | 9.98E+03 | |
| | 8644 | Lung | 14 days | 1.24E+04 | |
| | 8649 | Lung | 14 days | 4.96E+03 | |
| | 8652 | Lung | 14 days | 1.76E+03 | |
| | 8654 | Lung | 14 days | 5.37E+02 | |
| | 8657 | Lung | 14 days | 1.15E+04 | |
| Saline | 8631 | Lung | 2 days | 1.51E+06 | 2.90E+06 |
| | 8636 | Lung | 4 days | 5.68E+06 | |
| | 8645 | Lung | 3 days | 3.49E+06 | |
| | 8648 | Lung | 2 days | 2.39E+05 | |
| | 8650 | Lung | 2 days | 1.21E+06 | |
| | 8655 | Lung | 2 days | 5.26E+06 | |

at this time-point but 8 out of 12 animals treated with levofloxacin were positive at this same time. Moreover, when comparing only the two antibiotic-treated groups (to remove the high variability of the saline-treated group from the statistical analysis), the average level of bacteremia was significantly (p<0.05) lower among the animals treated with VIBATIV® (telavancin) (FIG. 5b). This would suggest that the VIBATIV® (telavancin) dose used for the study was more effective at eliminating the bacteria in the blood relative to the levofloxacin dose used. By 48 hours post-challenge, all antibiotic-treated animals were negative for bacteremia, and they remained so for the remainder of the post-challenge period. Overall, these results show that VIBATIV® (telavancin) was able to rapidly clear B. anthracis from circulation.

Although B. anthracis was also detected in the brains, mediastinal lymph nodes and spleens of the saline-treated control animals, no bacteria were recovered from these tissues from any animal in either of the antibiotic-treated groups (Table 8).

TABLE 8
B. anthracis bacterial load in saline-treated group

| | | | Average |
|---|---|---|---|
| | Group | | Bacterial Load |
| Group | size | Tissue | (cfu/g) |
| Saline | 6 | Brain | 6.68E+05 |
| | | Lymph Node | 2.59E+06 |
| | | Spleen | 6.43E+06 |

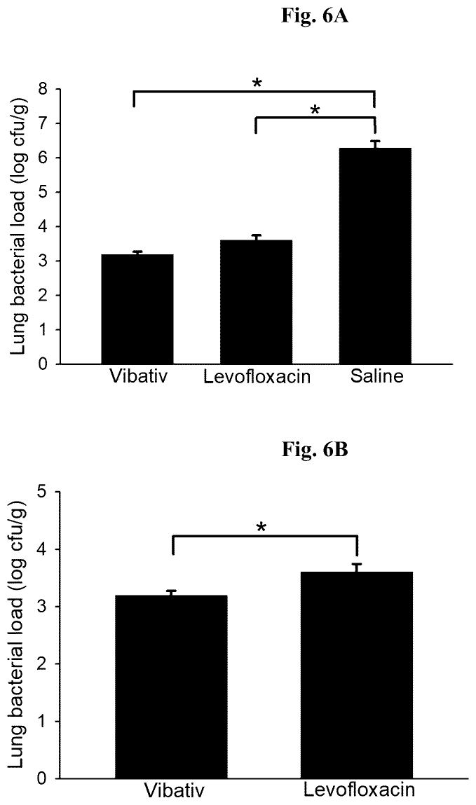

In comparison of B. anthracis bacterial loads in lung tissue, there were significantly (p<0.05) less bacteria in the two antibiotic-treated groups relative to the saline control group (FIG. 6a). When comparing only the two antibiotic-treated groups (to remove the high variability of the saline-treated group from the statistical analysis), the average bacterial load in the lung tissue was significantly (p<0.05) lower among the animals treated with VIBATIV® (telavancin) (primarily due to differences in female animals) (FIG. 6b), suggesting once again that the VIBATIV® (telavancin) dose used for the study was more effective at eliminating the bacteria in tissues relative to the levofloxacin dose used. Since tissues were not collected for assessment of bacterial load at various scheduled times post-challenge, it is not known whether VIBATIV® (telavancin) completely prevented the migration of B. anthracis to the tissues, or if it cleared the bacteria after the tissues were infected. Nonetheless, these results show that VIBATIV® (telavancin) can prevent, clear, and/or reduce B. anthracis infection of tissues after inhalation anthrax challenge.

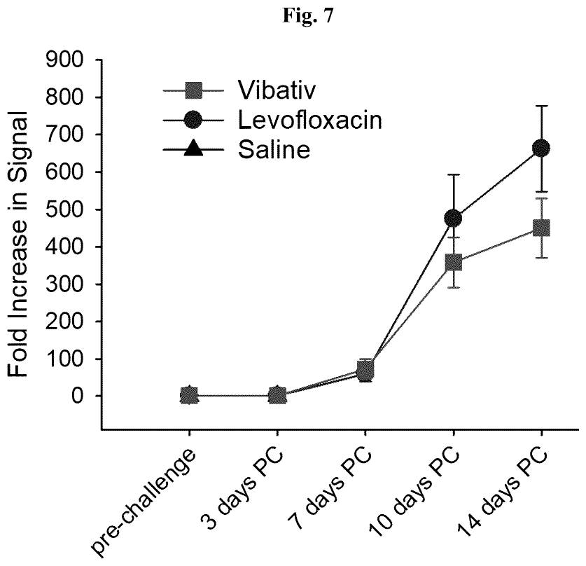

The animals in the two antibiotic-treated groups began to develop antibody responses to B. anthracis protective antigen as early as 7 days post-challenge, and the responses intensified at 10- and 14-days post-challenge (FIG. 7). There were no significant (p>0.05) differences in serum anti-PA IgG titers among the two antibiotic-treated groups; however, each antibiotic-treated group had a significant (p<0.05) increase over time. The negative control group, on the other hand, showed no increase in anti-PA IgG levels over time since they succumbed to the anthrax infection before the immune response could produce significant levels of serum anti-PA IgG (FIG. 7). These results illustrated that VIBATIV® (telavancin) did not alter the humoral immune response to PA generated following antibiotic treatment of anthrax infection.

At necropsy, the animals in the saline-treated group consistently showed blackened, enlarged, hemorrhagic mediastinal lymph nodes as well as discoloration and diffuse hemorrhage of the lungs with pleural effusion. Also evident was pericardial effusion and multifocal hemorrhage on the serosal surface of the vermiform appendix. Additional notable findings, although less consistent, in the saline-treated group were the presence of hemorrhage in the meninges, multifocal discoloration of the cecum, and hemorrhage in the thymus. Importantly, all these findings have been reported to be associated with anthrax infection in animal models. The animals treated with VIBATIV® (telavancin) and levofloxacin exhibited similar gross pathology. Even though all the antibiotic-treated animal survived the challenge, some of them still exhibited a few pathologic findings such as pulmonary congestion, multifocal hemorrhage of the lungs, and enlarged mediastinal lymph nodes, which is evidence of disease mitigation.

The saline-treated animals had lesions that were consistent with exposure to aerosolized anthrax and included cellular/tissue degeneration/necrosis, congestion, hemorrhage, edema, inflammatory infiltrates (fibrinous, mixed cellular), and/or the presence of bacteria in multiple tissues (particularly the mediastinal lymph node, spleen, and liver). The severity of the lesions varied from 1 to 4 of 4. All VIBATIV® (telavancin)- and levofloxacin-treated animals (both sexes) had mitigation of anthrax induced lesions, and differences in lesion mitigation between the two drugs were equivocal. These results show that VIBATIV® (telavancin) treatment reduces the pathology associated with anthrax infection.

### Example 3: Evaluation of Post-Exposure Delayed Administration of VIBATIV® (Telavancin) Against Inhalational Anthrax in a Rabbit Model

The basic study design is shown in Table 9. A total of 30 adult New Zealand White (NZW) rabbits were randomized on the basis of gender (50% males and 50% females) into the seven experimental groups listed in Table 9. There was an equal number of male and female rabbits per test group. All animals were challenged individually via muzzle-only aerosol with 200 LD₅₀ (2.0×10⁷ CFUs) of purified B. anthracis Ames spores (NR3838) using real-time plethysmography and a muzzle-only aerosol exposure chamber. Treatment commenced at various times after detection of protective antigen (PA) in sera. Blood samples were collected pre-challenge and at various times post-challenge for determination of bacterial/spore load. Furthermore, serum samples were collected at the various time-points to assess the levels of PA and anti-PA IgG.

TABLE 9
Experimental design

| | | Number of | | | Dose | |
|---|---|---|---|---|---|---|
| Group | Treatment | Animals | Dose & route | | Frequency | Initiation of Treatment |
| 1 | Vibativ | 3 | 30 | mg/kg IV | BID for | At + PA-ECL |
| 2 | | 9 | | | 5 days | At + PA-ECL + 18 hrs |
| 3 | | 3 | | | | At + PA-ECL + 36 hrs |
| 4 | Levofloxacin | 2 | 12.5 | mg/kg IV | SID for | At + PA-ECL |
| 5 | | 8 | | | 5 days | At + PA-ECL + 18 hrs |
| 6 | | 3 | | | | At + PA-ECL + 36 hrs |
| 7 | Saline | 2 | 0 | IV | SID for | At + PA-ECL |
| | | | | | 5 days | |

Daily clinical observations, which includes daily weights, were recorded on the day of challenge, to determine baseline measures/observations, and through the end of the designated in-life period to evaluate the progress of the infection and all clinical outcomes. Temperature was also recorded via implanted data loggers for each animal starting upon placement on the study and continued through the end of the in-life phase of the study. Survival was monitored for 14 days post-challenge, after which time all survivors were euthanized and necropsied, and terminal samples taken. Any animal found to be moribund or in respiratory distress prior to the end of the in-life phase was humanely euthanized and necropsied. Lastly, tissue samples (lung, mediastinal lymph nodes, brain, and spleen) were collected from all animals either at the time of euthanasia or at the end of the in-life period for determination of bacterial/spore load and histopathology (liver, kidneys, and heart were also collected for histopathology). If an animal was found dead, the time-to-death was determined as accurately as possible, and any terminal samples that could be recovered were taken.

A total of 30 NZW rabbits, 50% male and 50% female, were purchased from Covance (Denver, PA). A venous access port (VAP) was surgically implanted into the external jugular vein of each animal to facilitate both the collection of multiple blood specimens and the I.V. administration of the therapeutics during the study. For this study, the 30 animals were divided into two cohorts, which arrived four weeks apart. The animals were distributed into the seven experimental groups and two cohorts according to Table 10 (the distribution of animals for Cohort 2 was changed from the original study design. After arrival, the animals acclimated for at least 72 hours during which time each was given a thorough physical examination by a veterinarian.

TABLE 10
Group/cohort animal distribution

| Cohort | Group 1 | Group 2 | Group 3 | Group 4 | Group 5 | Group 6 | Group 7 | Total |
|---|---|---|---|---|---|---|---|---|
| 1 | 1 | 3 | 3 | 1 | 3 | 3 | 1 | 15 |
| | (1M, 0F) | (2M, 1F) | (1M, 2F) | (0M, 1F) | (2M, 1F) | (1M, 2F) | (1M, 0F) | (8M, 7F) |
| 2 | 2 | 6 | 0 | 1 | 5 | 0 | 1 | 15 |
| | (1M, 1F) | (2M, 4F) | (0M, 0F) | (1M, 0F) | (3M, 2F) | (0M, 0F) | (0M, 1F) | (7M, 8F) |
| Total | 3 | 9 | 3 | 2 | 8 | 3 | 2 | 30 |
| | (2M, 1F) | (4M, 5F) | (1M, 2F) | (1M, 1F) | (4M, 4F) | (2M, 1F) | (1M, 1F) | (15M, 15F) |

Following acclimation, each animal was implanted intraperitoneally with a temperature data logger for the purpose of recording the animals' temperature for the entire in-life period, after which the data were downloaded and analyzed. After 10-14 days of recovery, the animals were transferred to the facility where the aerosol challenge and all subsequent experimental procedures were performed.

Challenge was by aerosol with 200 LD₅₀ of purified B. anthracis Ames spores using an aerosol control platform (Biaera Technologies, Hagerstown, MD), fitted with a muzzle-only aerosol chamber using computer control of humidity, pressure, and air flow. Real-time plethysmography was performed on each rabbit using a pair of elastic band sensors (DSI, New Brighton, MN) placed around the animal's thorax and abdomen, which were then calibrated with a pneumotach fitted to the face of each animal. After calibration, the rabbit's muzzle was inserted into the aerosol chamber. The target dose of spores (D_{P}) for aerosol deposition in the lungs was 2.0×10⁷ cfu. The 6-jet Collison nebulizer used to generate the aerosols ideally yields a S_{f} of 1.0-2.0×10⁻⁶ for B. anthracis spores. The nebulizer concentration to deliver this dose was calculated using standard algorithms (Roy CJ, LML Pitt. 2005. Infectious disease aerobiology: Aerosol challenge methods. In: Biodefense: Research Methodology and Animal Models, Swearengen JL (Ed.). CRC Press, Boca Raton, FL), combined with a standard volume of air to deliver the target challenge dose of spores. Aerosol samples were collected continuously for confirming the challenge dose of spores for each animal by serial dilution and plating on blood agar plates. The duration of aerosol delivery was based on the respiration rate of each animal, plotted on the computer screen, and controlled by the aerosol system computer (Biaera Technologies, Hagerstown, MD).

Upon receipt, VIBATIV© (telavancin) was stored at 2-8° C., as directed by the manufacturer, with temperature monitoring. Levofloxacin, upon receipt, was stored at room temperature with temperature monitoring. For therapeutic treatment of Groups 1-3, the appropriate amount of VIBATIV® (telavancin) for each animal was freshly prepared daily by dissolving in sterile, pyrogen-free water to a concentration of 25 mg/ml and administered IV via the implanted VAP at 30 mg/kg BID (every 12 h) for 5 days (Tables 11a, 11b; o=Blood collection for bacteremia; §=Blood collection for antigenemia; #=Blood collection for Anti-PA IgG assessment; *=If necessary; Day 0=aerosol challenge; V=VIBATIV© (telavancin); L=Levofloxacin). Groups 4-6 (positive controls) received levofloxacin IV, via the VAP, at a concentration of 12.5 mg/kg once a day for 5 days. The appropriate dilutions of levofloxacin were prepared using sterile, pyrogen-free water. Lastly, Group 7 (negative control) received saline once a day for 5 days administered IV via the VAP. Treatments commenced either 0, 18, or 36 hours after detection of protective antigen (PA) in the animals' sera (within 4 hours after blood collection for antigenemia analysis).

The animals were not treated on an individual basis for the duration of the entire treatment period. The first dose administrations were prepared on an individual animal basis; however, all subsequent daily doses were prepared for treatment groups in the morning and delivered during the morning and/or night of each dosing day.

TABLE 11a
Therapeutic Dosing and Bleeding Schedule, Days −7 to 2.25

| | Days PC | | | | | | | | | | |
|---|---|---|---|---|---|---|---|---|---|---|---|
| | n | −7 | Challenge 0 | 0.5 | 0.75 | 1 | 1.25 | 1.5 | 1.75 | 2 | 2.25 |
| | Hours PC | | | | | | | | | | |
| | n | −168 | 0 | 12 | 18 | 24 | 30 | 36 | 42 | 48 | 54 |
| Vibativ | 3 | ◯ § # | — | ◯ § | V § | ◯ §* | §* | V §* | §* | V ◯ §* | |
| 30 mg/kg | | | | | | | | | | | |
| Vibativ | 9 | ◯ § # | — | ◯ § | § | ◯ §* | §* | V §* | §* | V ◯ §* | |
| 30 mg/kg | | | | | | | | | | | |
| Vibativ | 3 | ◯ § # | — | ◯ § | § | ◯ §* | §* | §* | §* | ◯ §* | V |
| 30 mg/kg | | | | | | | | | | | |
| Levofloxacin | 2 | ◯ § # | — | ◯ § | L § | ◯ §* | §* | §* | §* | L ◯ §* | |
| 12.5 mg/kg | | | | | | | | | | | |
| Levofloxacin | 8 | ◯ § # | — | ◯ § | § | ◯ §* | §* | L §* | §* | L ◯ §* | |
| 12.5 mg/kg | | | | | | | | | | | |
| Levofloxacin | 3 | ◯ § # | — | ◯ § | § | ◯ §* | §* | §* | §* | ◯ §* | L |
| 12.5 mg/kg | | | | | | | | | | | |
| Control | 2 | ◯ § # | — | ◯ § | S § | ◯ §* | §* | §* | §* | S ◯ §* | |
| (Saline) | | | | | | | | | | | |

TABLE 11b
Therapeutic Dosing and Bleeding Schedule, Days 2.5 to 14

| | Days PC | | | | | | | | | | | | |
|---|---|---|---|---|---|---|---|---|---|---|---|---|---|
| | n | 2.5 | 3 | 3.5 | 4 | 4.5 | 5 | 5.5 | 6 | 6.5 | 7 | 10 | 14 |
| | Hours PC | | | | | | | | | | | | |
| | n | 60 | 72 | 84 | 96 | 108 | 120 | 132 | 144 | 156 | 168 | 240 | 336 |
| Vibativ | 3 | V | V ◯ # | V | V ◯ | V | V ◯ | V | | | ◯ # | ◯ # | ◯ # |
| 30 mg/kg | | | | | | | | | | | | | |
| Vibativ | 9 | V | V ◯ # | V | V ◯ | V | V ◯ | V | V | | ◯ # | ◯ # | ◯ # |
| 30 mg/kg | | | | | | | | | | | | | |
| Vibativ | 3 | | V ◯ # | V | V ◯ | V | V ◯ | V | V | V | V ◯ # | ◯ # | ◯ # |
| 30 mg/kg | | | | | | | | | | | | | |
| Levofloxacin | 2 | | L ◯ # | | L ◯ | | L ◯ | | | | ◯ # | ◯ # | ◯ # |
| 12.5 mg/kg | | | | | | | | | | | | | |
| Levofloxacin | 8 | | L ◯ # | | L ◯ | | L ◯ | | | | ◯ # | ◯ # | ◯ # |
| 12.5 mg/kg | | | | | | | | | | | | | |
| Levofloxacin | 3 | | L ◯ # | | L ◯ | | L ◯ | | L | | ◯ # | ◯ # | ◯ # |
| 12.5 mg/kg | | | | | | | | | | | | | |
| Control | 2 | | S ◯ # | | S ◯ | | S ◯ | | | | ◯ # | ◯ # | ◯ # |
| (Saline) | | | | | | | | | | | | | |

Depending on the time of the initial treatment, the second treatment doses occurred as outlined in Table 12. This was based on a projected average challenge time of 10 a.m.

TABLE 12
Example Schedule for initial two treatment doses

| | | Time period | |
|---|---|---|---|
| Treatment | Time of blood | of initial | Time of second |
| groups | collection | treatment¹ | treatment |
| Vibativ | 4 AM | 6 AM-8 AM | Same calendar day |
| | | | 10 PM |
| | 10 AM | 12 PM-2 PM | Same calendar day |
| | | | 10 PM |
| | 4 PM | 6 PM-8 PM | Next calendar day |
| | | | 10 AM |
| | 10 PM | 12 AM-2 AM | Same calendar day |
| | | | 10 AM |
| Levofloxacin | 4 AM | 6 AM-8 AM | Next calendar day |
| | | | 10 AM |
| | 10 AM | 12 PM-2 PM | Next calendar day |
| | | | 10 AM |
| | 4 PM | 6 PM-8 PM | Next calendar day |
| | | | 10 AM |
| | 10 PM | 12 AM-2 AM | Same calendar day |
| | | | 10 AM |

Blood specimens were collected from each animal, via the VAP, prior to challenge and at specified times post-challenge (Tables 11a, b). Blood specimens were also collected from peripheral vessels in the ear (central artery or marginal vein). This alternate method of blood collection was proposed because B. anthracis in the blood of bacteremic animals has the potential to colonize the VAP (or catheter), thereby contaminating successive samples drawn from the port. The impact of catheter colonization can result in reporting an animal as bacteremia positive even though the blood has been cleared of bacteria by therapeutic treatment. Therefore, blood specimens used for the purpose of assessing bacteremia beginning 7 days post-challenge were collected from a peripheral blood vessel in the ear. Blood specimens were collected into 0.5 ml blood collection microtubes (Wampole Laboratories, Cranbury, NJ) for quantitative bacterial plate counts at the specified time-points (Tables 11a, b). Blood was also collected in serum separator microtubes (1.5 ml) at selected times pre- and post-challenge and the serum used for PA-ECL and anti-PA IgG titration (Tables 11a, b). Sera for IgG testing were stored frozen (−20° C.) and samples tested as a batch at the conclusion of the in vivo portion of the study. Approximately 2.0 ml of whole blood was collected from each animal at the collection times specified above for the various assays.

Bacterial concentration in the blood was determined using an automatic serial diluter and plater (easySpiral Dilute; Interscience). Whole blood, diluted in sterile water, was plated onto trypticase soy agar plates containing 5% sterile sheep blood (TSAB) and incubated at 37° C. for 16-24 hours. Colonies from the plates were then enumerated using an automatic colony counter (Scan 500; Interscience). Bacterial colonies having morphology typical of B. anthracis were subcultured and confirmed as B. anthracis with bacteriophage y.

Bacterial/spore load was also determined in lung, lymph node (mediastinal), brain, and spleen. These tissues were homogenized in sterile water using a Stomacher 80 MicroBiomaster (Seward Ltd), and the homogenate was serially diluted in PBS or water and plated onto TSAB plates using the automatic diluter/plater and incubated at 37° C. for 16-24 hours. Colonies from the plates were then enumerated using an automatic colony counter (Scan 500; Interscience), and the bacterial load was presented as cfu per gram of tissue. Bacterial colonies having morphology typical of B. anthracis were subcultured and confirmed as B. anthracis with bacteriophage y.

B. anthracis PA was measured in serum using a rapid PA-ECL screening assay (MesoScale Discovery; Gaithersburg, MD). This assay kit utilizes a detection antibody in combination with specialized 96-well microtiter plates that contain electrodes coated with an anti-PA capture antibody to detect and/or quantify PA. Following processing and assay execution, the amount of light emitted in sample wells is used to directly measure the amount of PA present in the serum based on a recombinant PA standard curve run in parallel. To quantitate the levels of PA in each serum sample, a standard curve (0-100 ng/ml) was analyzed in parallel on each assay day. Test samples were assayed in duplicate. The concentration of each test sample was extrapolated from the standard curve. Values that fell below the lower limits of the standard curve were defined as LLOQ (lower limit of quantitation).

Anti-PA IgG was measured in serum via ECL similar to the PA-ECL screening assay. Biotinylated recombinant PA63 was bound to streptavidin-coated plates (MSD) and used as the capture antigen. Detection was accomplished using SULFO-TAG labeled anti-rabbit antibody and read buffer (MSD). Results were presented as fold-increase in signal relative to the pre-challenge serum samples.

Necropsies were performed by veterinarians on animals that either succumbed to infection or were euthanized. In addition to gross pathology, selected tissue fixation, embedding, sectioning, and staining was performed for microscopic pathology. Histopathology was performed on H&E-stained sections of lung, lymph nodes (mediastinal), brain, spleen, liver, kidneys, and heart.

During the in-life phase of the study, clinical observations were performed/recorded at least twice daily, with more observations being performed during the peak time of infection when the animals showed increased signs of infection. The animals' weights were recorded daily starting upon placement on the study and continued through the end of the in-life period of the study. Any animal found to be immobile (severe lack of movement in response to stimulation), unable to get to food/water, and/or in respiratory distress (abdominal breathing, open mouth breathing, nasal flaring) was immediately euthanized, and the time of death was recorded. At the time of euthanasia, all terminal samples were also collected. If an animal was found dead, the time of death was determined as accurately as possible, and any terminal samples able to be recovered were taken. Lastly, all survivors were humanely euthanized on the last day of the in-life period, and all terminal samples taken.

A randomized block design was designed and conducted. Blocks (gender) were males and females. There were the five treatments as described above (only the groups having animals that began treatment were analyzed for statistical comparisons). Male and female rabbits (experimental units within blocks) were randomized to the indicated treatment groups. Because of the relatively high and imbalanced animal loss, which occurred throughout the experiment, animal gender was necessarily disregarded in the analyses. Animal death rates were analyzed using contingency tables and either Chi Square analyses or Fishers Exact Tests to compare the treatment groups. For measurement variables, Analysis of Variance (ANOVA) methods appropriate for a completely randomized experiment were used. Specific treatment differences were compared and tested using pooled inter-animal variability as the error variance. Since means and standard deviations were generally correlated, as expected, the response measures were transformed to their rank scores before analysis. Significance levels for directional hypotheses were used.

During aerosol exposure, the rabbits' respiration was monitored/recorded in real-time using a DSI plethysmography system that was integrated with the Biaera aerosol control platform. For each animal, a target accumulated total volume (ATV) was set to 7,000 ml which was predetermined to give a presented dose (D_{P}) of 2.0×10⁷ spores. The D_{P} denotes the number of spores deposited into the animals' lungs. As soon as the animal inhaled 7,000 ml of air, the aerosol exposure to spores was terminated by the computer. The aerosol exposure times among all animals in the two cohorts ranged from 5 to 31 min (Table 14a & b). The range of exposure times for both cohorts was due to the animals' varying respiration intensities that resulted from their differing levels of sedation during the aerosol challenge. Nonetheless, despite the various exposure times, all animals were challenged with comparable challenge doses due to the capabilities of the real-time plethysmography system.

The cumulative mean D_{P} for the two cohorts was 2.46×10⁷ spores (target was 2.0×10⁷ spores) which is 246 LD₅₀. The overall mean S_{F} was 2.04×10⁻⁶. S_{F}, which conveys the efficiency of each aerosol spray, is a unitless parameter defined as the spore concentration of the aerosol (C_{AERO}) divided by the spore concentration of the nebulizer suspension (C_{NEB}). A mean S_{F} of 2.04×10⁻⁶ indicates an overall high efficiency of aerosolization.

Treatments were prepared and administered at specified times after detection of PA in the animals' sera. Table 13 provides the serum PA concentration for each animal and shows the time-point at which PA was first detected. PA was detected as early as 18 hours post-challenge and as late as 30 hours post-challenge (PC), while the majority of animals were positive for PA at 18 to 24 hours post-challenge. The concentration of PA in the sera ranged from approximately 130 to 3,100 μg/ml. Value reported as <LLOQ (lower limit of quantitation) denotes measurements that were below the standard curve.

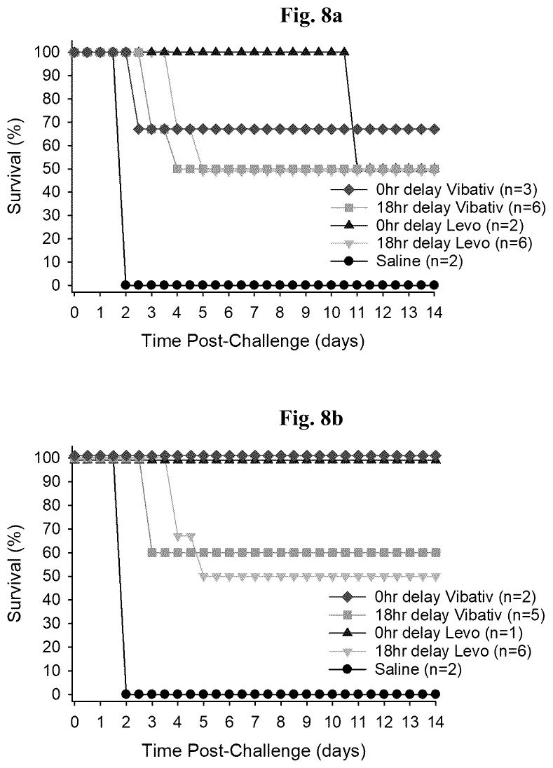

FIG. 8a shows the survival results for the treatment groups over the 14-day post-challenge period. None of the animals that were to begin treatment at 36 hr post-antigenemia survived to receive the initial treatment; therefore, those animals were excluded from all comparative analyses in this study report. The 36 hr delayed treatment occurred only with Cohort 1, and the animals that would have been used for the 36 hr delayed treatment in Cohort 2 were used instead for the 18 hr delayed treatment groups. Overall, VIBATIV® (telavancin) and levofloxacin protected similarly among the treatment groups. The groups given VIBATIV® (telavancin) and levofloxacin at the time of antigenemia each had one animal (Animals 6083 and 6063) succumb during the post-challenge period resulting in overall survival rates of 67% and 50%, respectively; however, both these animals may have succumbed

TABLE 13
B. anthracis antigenemia levels

| | Animal | −7 | 12 h | 18 h | 24 h | 30 h |
|---|---|---|---|---|---|---|
| Group | ID# | days | PC | PC | PC | PC |
| Vibativ + | 6062 | 0 | 0 | 501 | | |
| PA | 6068 | 0 | 0 | 1,550 | | |
| | 6083 | 0 | 0 | 1,691 | | |
| Vibativ + | 6060 | 0 | 0 | 128 | | |
| PA + 18 h | 6064 | 0 | 0 | <LLOQ | 589 | |
| | 6066 | 0 | 0 | 0 | 111 | |
| | 6069 | 0 | 0 | 1,115 | | |
| | 6072 | 0 | 0 | 541 | | |
| | 6074 | 0 | 0 | 142 | | |
| | 6076 | 0 | 0 | 0 | <LLOQ | 431 |
| | 6086 | 0 | 0 | <LLOQ | 1,240 | |
| | 6089 | 0 | 0 | <LLOQ | 827 | |
| Vibativ + | 6078 | 0 | 0 | <LLOQ | 1,367 | |
| PA + 36 h | 6080 | 0 | 0 | 474 | | |
| | 6082 | 0 | 0 | 603 | | |
| Levo + PA | 6063 | 0 | 0 | 0 | 0 | 13 |
| | 6075 | 0 | 0 | 2,825 | | |
| Levo + | 6059 | 0 | 0 | <LLOQ | 3,079 | |
| PA + 18 h | 6061 | 0 | 0 | 0 | 331 | |
| | 6065 | 0 | 0 | 0 | <LLOQ | 510 |
| | 6071 | 0 | 0 | 0 | 0 | 652 |
| | 6073 | 0 | 0 | <LLOQ | 689 | |
| | 6077 | 0 | <LLOQ | 2,746 | | |
| | 6079 | 0 | 0 | <LLOQ | 2,075 | |
| | 6088 | 0 | 0 | 0 | 0 | 618 |
| Levo + | 6081 | 0 | 0 | 2,180 | | |
| PA + 36 h | 6084 | 0 | 0 | <LLOQ | 2,446 | |
| | 6087 | 0 | 0 | 0 | 1,023 | |
| Saline | 6067 | 0 | 0 | 250 | | |
| | 6085 | 0 | 0 | 532 | | |

to conditions other than anthrax infection, or other factors may have contributed to death. In the case of Animal 6083 (VIBATIV® (telavancin)-treated), there was an overgrowth of another bacterium in the lungs, lymph nodes, and spleen, while B. anthracis could not be detected in these tissues. In the case of Animal 6063 (levofloxacin-treated), the animal succumbed on Day 11 post-challenge which is an uncommonly extended time-to-death in the rabbit model of inhalation anthrax. Furthermore, no B. anthracis was detected in the blood, brain, lymph nodes, and spleen after plating for bacterial load, which historically is typical for animals that are protected against anthrax infection and survive the lethal anthrax challenge. Moreover, this animal did not develop an antibody response to PA by Day 10 post-challenge which is abnormal in this model of infection. For these reasons, in addition to the fact that the treatment doses of VIBATIV® (telavancin) and levofloxacin used in this study were previously shown to be fully protective if administered at the time of antigenemia, the survival analysis (and subsequent analyses) was also performed without Animals 6083 and 6063 (FIG. 8b). Animal 6066 in the 18 hr delay VIBATIV® (telavancin) group was also excluded from data presented in FIG. 8b since, like Animal 6083, there was an overgrowth of another bacterium in the animal's lungs, lymph nodes, and spleen with no B. anthracis detectable in these tissues or the blood. Even with these three animals (6083, 6063, and 6066) excluded, VIBATIV® (telavancin) and levofloxacin still conveyed similar levels of protection against anthrax infection among the treatment groups (FIG. 8b), with both groups administered VIBATIV® (telavancin) and levofloxacin at the time of antigenemia showing 100% survival, and the groups administered VIBATIV® (telavancin) and levofloxacin 18 hr post-antigenemia showing 60% and 50% survival, respectively. There was no survival among the group given saline. Unfortunately, due to the small sample sizes, no statistically significant differences were detected among the groups. Overall, the results show that delaying VIBATIV® (telavancin) treatment still conveys partial protection against inhalation anthrax that is comparable to the level of protection provided by delayed treatment with levofloxacin.

The antibiotic-treated groups (VIBATIV® (telavancin) and levofloxacin) exhibited comparable temperature responses after challenge. Specifically, animals in these groups had febrile responses at approximately Day 1 post-challenge that rose above 40.5° C. before Day 2. The decline in temperature from Days 2-4 for the 18 hr delayed antibiotic-treated groups was likely due to the loss of animals that had succumbed to infection, but the remaining animals in these two groups continued to be febrile as indicated by elevated temperatures at Day 5. The temperatures returned to baseline by Day 6. The groups treated with antibiotics at the time of antigenemia only showed the initial febrile response at Day 1 post-challenge. The group treated with saline also exhibited a fever at Day 1, but both the animals succumbed prior to Day 2. These results show that when survival was achieved, delayed treatment with VIBATIV® (telavancin) was effective at reducing the febrile response that is consistently reported with anthrax infection.

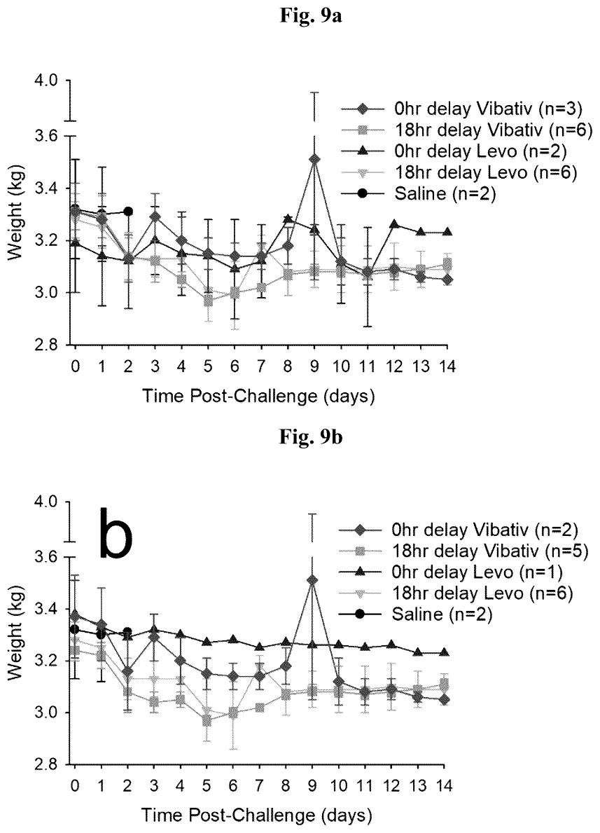

FIG. 9 shows the average daily weights for each treatment group during the post-challenge period with (FIG. 9a) and without (FIG. 9b) Animals 6083, 6063, and 6066. The average weights for all antibiotic-treated groups began to drop slightly after challenge. The average weights rebounded for the groups treated with either VIBATIV® (telavancin) or levofloxacin at the time of antigenemia, but the average weights continued to decline until Day 5-6 for the groups treated with antibiotics beginning 18 hr post-antigenemia. This decline among the delayed treatment groups may have been due primarily to the weight loss among the animals that did not survive the challenge. The average daily weight of the saline-treated group also showed a slight decline prior to the two animals succumbing to infection. With regards to the maximum weight reduction over the post-challenge period, there were no significant differences between the groups. Weight reduction is commonly seen in the rabbit model of inhalation anthrax infection, but this sequela appears to be remedied with VIBATIV® (telavancin) treatment as the antibiotic clears the infection.

Table 14 indicates the B. anthracis bacterial load in the blood for each animal in all the original treatment groups (including the 36 hr treatment delay groups). Aside from Animal 6083, the animals in the groups administered VIBATIV® (telavancin) and levofloxacin at the time of antigenemia never developed bacteremia at a detectable level. On the other hand, bacteremia was detected as early as 24 hr post-challenge in the groups wherein treatment was delayed as well as in the saline control group. The level of bacteremia reached 10⁴ pfu/ml in the delayed treatment groups and 105 pfu/ml in the saline control group, and the bacteremia cleared by either 48, 72, or 96 hr post-challenge in the treated animals that survived the post-challenge period. Obviously, the rate at which the bacteremia cleared was dependent on how soon treatment was initiated, but there was no significant difference in the level of bacteremia between the delayed treatment groups even when comparing the level of bacteremia at approximately 24 hr post-treatment initiation for the groups. Interestingly, a few of the animals treated with VIBATIV® (telavancin) beginning 18 hr post-antigenemia succumbed to infection despite them achieving an undetectable concentration of bacteria in their blood. This occurred in a few of the animals treated with levofloxacin beginning at 18 hr post-antigenemia as well. Overall, these results show that VIBATIV® (telavancin) was able to rapidly clear B. anthracis from circulation although delaying treatment prolonged bacterial clearance and increased mortality.

TABLE 14
B. anthracis bacteremia levels presented as cfu/ml

| | Animal | −7 | 12 h | 24 h | 48 h | 72 h | 96 h | 120 h | 7 days | 10 days | 14 days |
|---|---|---|---|---|---|---|---|---|---|---|---|
| Group | ID# | days | PC | PC | PC | PC | PC | PC | PC | PC | PC |
| Vibativ + | 6062* | 0 | 0 | 0 | 0 | 0 | 0 | 0 | 0 | 0 | 0 |
| PA | 6068* | 0 | 0 | 0 | 0 | 0 | 0 | 0 | 0 | 0 | 0 |
| | 6083* | 0 | 0 | 0 | 3 × 10¹ | | | | | | |
| Vibativ + | 6060* | 0 | 0 | 8.86 × 10³ | 0 | | | | | | |
| PA + 18 h | 6064* | 0 | 0 | 0 | 0 | | | | | | |
| | 6066* | 0 | 0 | 0 | 0 | 0 | 0 | | | | |
| | 6069 | 0 | 0 | 1.03 × 10⁴ | | | | | | | |
| | 6072* | 0 | 0 | 5.44 × 10³ | 0 | 0 | 0 | 0 | 0 | 0 | 0 |
| | 6074 | 0 | 0 | 3.06 × 10³ | | | | | | | |
| | 6076* | 0 | 0 | 0 | 5.70 × 10³ | 2.00 × 10¹ | 0 | 0 | 0 | 0 | 0 |
| | 6086* | 0 | 0 | 0 | 0 | 0 | 0 | 0 | 0 | 0 | 0 |
| | 6089 | 0 | 0 | 1.02 × 10³ | | | | | | | |
| Vibativ + | 6078 | 0 | 0 | 9.79 × 10⁵ | | | | | | | |
| PA + 36 h | 6080 | 0 | 0 | 6.49 × 10⁴ | | | | | | | |
| | 6082 | 0 | 0 | 8.33 × 10⁴ | | | | | | | |
| Levo + | 6063* | 0 | 0 | 0 | 0 | 0 | 0 | 0 | 0 | 0 | |
| PA | 6075* | 0 | 0 | 0 | 0 | 0 | 0 | 0 | 0 | 0 | 0 |
| Levo + | 6059* | 0 | 0 | 6.58 × 10² | 0 | 0 | 0 | 0 | 0 | 0 | 0 |
| PA + 18 h | 6061 | 0 | 0 | 1.19 × 10² | | | | | | | |
| | 6065* | 0 | 0 | 1.33 × 10¹ | 2.18 × 10⁴ | 0 | 0 | | | | |
| | 6071* | 0 | 0 | 0 | 5.50 × 10³ | 0 | 0 | 0 | 0 | 0 | 0 |
| | 6073 | 0 | 0 | 0 | | | | | | | |
| | 6077* | 0 | 0 | 1.37 × 10⁴ | 0 | 0 | 0 | | | | |
| | 6079* | 0 | 0 | 5.14 × 10¹ | 0 | 0 | | | | | |
| | 6088* | 0 | 0 | 0 | 3.00 × 10¹ | 0 | 0 | 0 | 0 | 0 | 0 |
| Levo + | 6081 | 0 | 0 | 1.25 × 10⁵ | | | | | | | |
| PA + 36 h | 6084 | 0 | 0 | 1.08 × 10⁵ | | | | | | | |
| | 6087 | 0 | 0 | 1.61 × 10⁴ | | | | | | | |
| Saline | 6067 | 0 | 0 | 1.27 × 10¹ | | | | | | | |
| | 6085 | 0 | 0 | 1.85 × 10⁵ | | | | | | | |

*Animals that began treatment

Tables 15a-d gives the tissue bacterial loads just for the animals that survived long enough to begin treatment. The animals that were treated with VIBATIV® (telavancin) or levofloxacin and survived the post-challenge period (i.e., the animals whose tissues were collected at 14 days post-challenge) had B. anthracis only in lung tissue, and the concentration ranged from 103 to 10⁴ cfu/g. However, there were no significant differences in lung bacterial loads between the treatment groups. In most cases, the animals that were treated with VIBATIV® (telavancin) or levofloxacin, yet succumbed to infection, also had B. anthracis present in the brain, but B. anthracis was never detected in the mediastinal lymph nodes and spleen of the antibiotic-treated animals. Values were not determined for the lung, lymph node, and spleen of Animals 6083 and 6066 due to overgrowth of another bacterium. These results show that VIBATIV® (telavancin) was still able to clear tissues of B. anthracis similar to levofloxacin, even when treatment was delayed.

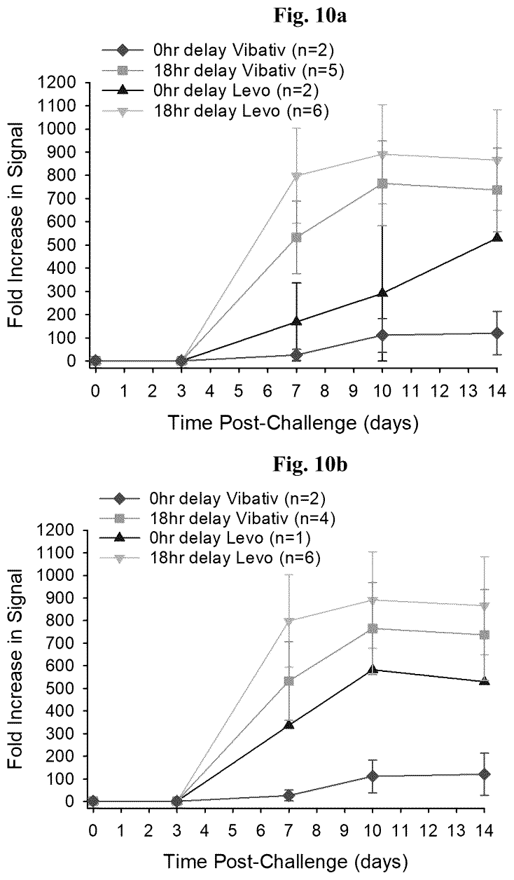

FIGS. 10a&b shows the average anti-PA antibody responses of the antibiotic-treated groups post-challenge. These results included only animals that survived to at least Day 3 post-challenge since Day 0 was used at the reference time-point and Day 3 was the first time-point for antibody assessment (FIG. 10b also excludes Animals 6063 and 6066). There was an increase in average antibody response among the surviving animals in each treatment group, but the largest increases were exhibited by the groups treated with VIBATIV® (telavancin) and levofloxacin beginning 18 hr post-antigenemia. These increases in antibody response for the 18 hr delayed groups were significantly (p<0.05) elevated from Day 3 to Day 7; however, there were no significant differences between Days 7, 10, and 14 (statistical comparisons with the 0 hr delayed treatment groups were not performed due to insufficient sample sizes). Interestingly, the group treated with VIBATIV® (telavancin) at the time of antigenemia exhibited a relatively low fold increase in antibody response, possibly due to a low concentration of serum PA, the primary immunogen, that could have resulted from the effective clearing of B. anthracis from the blood/tissues by VIBATIV® (telavancin). In general, these results illustrated that delayed VIBATIV® (telavancin) treatment did not alter the humoral immune response to PA generated after antibiotic treatment of anthrax infection.

TABLE 15a
B. anthracis bacterial load in brain tissue

| | | Time of | |
|---|---|---|---|
| | | tissue | |
| | | collection | |
| | Animal | (time post- | Bacterial |
| Group | ID# | challenge) | Load (cfu/g) |
| Vibativ + | 6062 | 14 days | 0 |
| PA | 6068 | 14 days | 0 |
| | 6083 | 3 days | 3.00 × 10⁵ |
| Vibativ + | 6060 | 3 days | 2.27 × 10³ |
| PA + 18 h | 6064 | 3 days | 5.08 × 10³ |
| | 6066 | 4 days | 4.92 × 10² |
| | 6072 | 14 days | 0 |
| | 6076 | 14 days | 0 |
| | 6086 | 14 days | 0 |
| Levo + PA | 6063 | 12 days | 0 |
| | 6075 | 14 days | 0 |
| Levo + | 6059 | 14 days | 0 |
| PA + 18 h | 6065 | 5 days | 0 |
| | 6071 | 14 days | 0 |
| | 6077 | 4 days | 4.12 × 10¹ |
| | 6079 | 4 days | 9.01 × 10² |
| | 6088 | 14 days | 0 |
| Saline | 6067 | 2 days | 7.84 × 10⁵ |
| | 6085 | 2 days | 1.46 × 10⁵ |

TABLE 15b
B. anthracis bacterial load in lung tissue

| | | Time of | |
|---|---|---|---|
| | | tissue | |
| | | collection | |
| | Animal | (time post- | Bacterial |
| Group | ID# | challenge) | Load (cfu/g) |
| Vibativ + PA | 6062 | 14 days | 7.08 × 10³ |
| | 6068 | 14 days | 1.34 × 10⁴ |
| | 6083 | 3 days | ND |
| Vibativ + | 6060 | 3 days | 3.28 × 10⁴ |
| PA + 18 h | 6064 | 3 days | 8.19 × 10⁴ |
| | 6066 | 4 days | ND |
| | 6072 | 14 days | 1.63 × 10⁴ |
| | 6076 | 14 days | 5.81 × 10³ |
| | 6086 | 14 days | 5.72 × 10³ |
| Levo + PA | 6063 | 12 days | 1.05 × 10³ |
| | 6075 | 14 days | 1.68 × 10⁴ |
| Levo + | 6059 | 14 days | 1.05 × 10³ |
| PA + 18 h | 6065 | 5 days | 6.13 × 10³ |
| | 6071 | 14 days | 1.62 × 10⁴ |
| | 6077 | 4 days | 5.68 × 10⁴ |
| | 6079 | 4 days | 1.68 × 10⁴ |
| | 6088 | 14 days | 1.22 × 10⁴ |
| Saline | 6067 | 2 days | 4.41 × 10⁷ |
| | 6085 | 2 days | 5.18 × 10⁶ |

TABLE 15c
B. anthracis bacterial load in lymph node tissue

| | | Time of | |
|---|---|---|---|
| | | tissue | |
| | | collection | |
| | Animal | (time post- | Bacterial |
| Group | ID# | challenge) | Load (cfu/g) |
| Vibativ + PA | 6062 | 14 days | 0 |
| | 6068 | 14 days | 0 |
| | 6083 | 3 days | ND |
| Vibativ + | 6060 | 3 days | 0 |
| PA + 18 h | 6064 | 3 days | 0 |
| | 6066 | 4 days | ND |
| | 6072 | 14 days | 0 |
| | 6076 | 14 days | 0 |
| | 6086 | 14 days | 0 |
| Levo + PA | 6063 | 12 days | 0 |
| | 6075 | 14 days | 0 |
| Levo + | 6059 | 14 days | 0 |
| PA + 18 h | 6065 | 5 days | 0 |
| | 6071 | 14 days | 0 |
| | 6077 | 4 days | 0 |
| | 6079 | 4 days | 0 |
| | 6088 | 14 days | 0 |
| Saline | 6067 | 2 days | 4.51 × 10⁶ |
| | 6085 | 2 days | 1.98 × 10⁷ |

TABLE 15d
B. anthracis bacterial load in spleen tissue

| | | Time of | |
|---|---|---|---|
| | | tissue | |
| | | collection | |
| | Animal | (time post- | Bacterial |
| Group | ID# | challenge) | Load (cfu/g) |
| Vibativ + PA | 6062 | 14 days | 0 |
| | 6068 | 14 days | 0 |
| | 6083 | 3 days | ND |
| Vibativ + | 6060 | 3 days | 0 |
| PA + 18 h | 6064 | 3 days | 0 |
| | 6066 | 4 days | ND |
| | 6072 | 14 days | 0 |
| | 6076 | 14 days | 0 |
| | 6086 | 14 days | 0 |
| Levo + PA | 6063 | 12 days | 0 |
| | 6075 | 14 days | 0 |
| Levo + | 6059 | 14 days | 0 |
| PA + 18 h | 6065 | 5 days | 0 |
| | 6071 | 14 days | 0 |
| | 6077 | 4 days | 0 |
| | 6079 | 4 days | 0 |
| | 6088 | 14 days | 0 |
| Saline | 6067 | 2 days | 1.71 × 10⁷ |
| | 6085 | 2 days | 1.78 × 10⁶ |

At necropsy, the animals in the saline-treated group showed diffuse, blackened lungs with thoracic effusion. Also present were diffuse meningeal red to black discolorations and petechial hemorrhage of the vermiform appendix. The animals that succumbed to infection despite being treated with VIBATIV® (telavancin) or levofloxacin also showed most of the gross pathology typically associated with fatal anthrax infection in the rabbit model including cerebral hemorrhage, diffused blackened lungs, enlarged blackened mediastinal lymph nodes, serosanguinous effusions in the thoracic cavity, diffuse discoloration of the liver, red discoloration of the trachea, and red/black discoloration of the vermiform appendix. Conversely, the surviving animals treated with VIBATIV® (telavancin) and levofloxacin either at the time of antigenemia or 18 hr post-antigenemia only exhibited diffuse dark red lungs and mild enlargement of the mediastinal lymph nodes. The reduced pathology with the surviving animals serves as evidence of disease mitigation.

The saline-treated animals had lesions that were consistent with exposure to aerosolized anthrax which included tissue degeneration/necrosis, the presence of bacteria (consistent with anthrax bacilli), fibrin, infiltrates, and hemorrhage. The severity of the lesions varied from 1 to 4 of 4 (1=minimal, 2=mild, 3=moderate, 4=marked). Anthrax-related lesions were generally mitigated in the surviving animals administered VIBATIV® (telavancin) or levofloxacin at the time of antigenemia or 18 hr post-antigenemia, but the severity of lesions in the treated animals that succumbed to infection was similar to that of the saline control animals. Interestingly, Animals 6066 and 6083, which are two of the three animals excluded due to the presence of another bacterium in their tissues, had severe lesions in the CNS. This suggests that the treatment was successful in reducing B. anthracis bacteremia, but treatment did not appear to fully prevent the development of inflammation. Overall, these results show that delayed VIBATIV® (telavancin) treatment can still reduce anthrax-related pathology, albeit to a much lesser extent compared to when the treatment is administered as soon as antigenemia develops.

We claim:

1. A method for treating a Bacillus anthracis (anthrax) infection in a subject, comprising administering to the subject a pharmaceutical composition consisting of a therapeutically effective amount of telavancin or a pharmaceutically acceptable salt thereof and at least one pharmaceutical excipient.

2. The method of claim 1, wherein the telavancin or pharmaceutically acceptable salt thereof is telavancin hydrochloride.

3. The method of claim 1, wherein the at least one pharmaceutical excipient is selected from the group consisting of 2-hydroxypropyl-β-cyclodextrin, mannitol, sodium hydroxide, hydrochloric acid, 5% Dextrose Injection, Sterile Water for Injection, 0.9% Sodium Chloride Injection, and combinations thereof.

4. The method of claim 1, wherein pharmaceutical composition is administered to the subject within 15 minutes of diagnosis of Bacillus anthracis infection or exposure to Bacillus anthracis.

5. The method of claim 1, wherein pharmaceutical composition is administered to the subject within 48 hours of diagnosis of Bacillus anthracis infection or exposure to Bacillus anthracis.

6. The method of claim 1, wherein the pharmaceutical composition is administered intravenously to the subject.

7. The method of claim 1, wherein the therapeutically effective amount is from 7.5 mg/kg to 15 mg/kg body weight of the subject.

8. The method of claim 1, wherein the pharmaceutical composition is administered once daily to the subject.

9. The method of claim 1, wherein the subject is a mammal.

10. The method of claim 1, wherein the Bacillus anthracis is a vegetative form of B. anthracis, a B. anthracis spore or a mixture of both.

11. The method of claim 1, wherein the Bacillus anthracis infection is selected from the group consisting of cutaneous anthrax, gastrointestinal anthrax, inhalational anthrax, or injection anthrax.

12. The method of claim 1, wherein the telavancin or a pharmaceutically acceptable salt thereof is the only antibiotic administered to the subject for treating the Bacillus anthracis infection.

13. A method for preventing a Bacillus anthracis (anthrax) infection in a subject at risk of exposure to Bacillus anthracis, comprising administering to the subject a pharmaceutical composition consisting of a therapeutically effective amount of telavancin or a pharmaceutically acceptable salt thereof and at least one pharmaceutical excipient.

14. The method of claim 13, wherein the pharmaceutical composition is administered less than 24 hours prior to the risk of exposure.

15. The method of claim 13, wherein the pharmaceutical composition is administered less than 15 days prior to the risk of exposure.

16. A method for treating or preventing a Bacillus anthracis (anthrax) infection in a mammal, comprising intravenously administering to the mammal a pharmaceutical composition comprising a therapeutically effective amount of telavancin hydrocholoride and at least one pharmaceutical excipient selected from the group consisting of 2-hydroxypropyl-β-cyclodextrin, mannitol, sodium hydroxide, hydrochloric acid, 5% Dextrose Injection, Sterile Water for Injection, 0.9% Sodium Chloride Injection, and combinations thereof.

17. The method of claim 16, wherein the Bacillus anthracis infection is selected from the group consisting of cutaneous anthrax, gastrointestinal anthrax, inhalational anthrax, or injection anthrax.

18. The method of claim 16, wherein the Bacillus anthracis is a vegetative form of B. anthracis, a B. anthracis spore or a mixture of both.

19. The method of claim 16, wherein the telavancin hydrochloride is the only antibiotic administered to the mammal for the treatment or prevention of the Bacillus anthracis infection.

20. The method of claim 16, wherein the pharmaceutical composition is administered once daily to the mammal.
